(19) United States
(12) Patent Application Publication
(10) Pub. No.: US 2024/0052028 A1
(43) Pub. Date: Feb. 15, 2024

(54) ENHANCEMENT OF ANTI-TUMOR PHAGOCYTOSIS

(71) Applicant: The Board of Trustees of the Leland Stanford Junior University, Stanford, CA (US)

(72) Inventors: Michael C. Bassik, Stanford, CA (US); Roarke Kamber, Stanford, CA (US)

(21) Appl. No.: 18/030,223

(22) PCT Filed: Oct. 5, 2021
(86) PCT No.: PCT/US2021/053614

§ 371 Date: Apr. 4, 2023

Related U.S. Application Data
(60) Provisional application No. 63/087,625, filed on Oct. 5, 2020.

(51) Int. Cl. C07K 16/28; A61K 31/513; A61K 45/06; C12Q 1/6869; C12Q 1/6886; C07K 14/705; C12Q 1/6806; C12N 5/078; A61P 35/00

(52) U.S. Cl. CPC C07K 16/2803; A61K 31/513; A61K 45/06; C12Q 1/6869; C12Q 1/6886; C07K 14/70596; C12Q 1/6806; C12N 5/0634; A61P 35/00; C07K 16/2896; A61K 2039/505

(57) ABSTRACT

The present disclosure provides methods for treating a disease or disorder or sensitizing a cell to phagocytosis. The methods comprise contacting a cell with an inhibitor of Adipocyte Plasma Membrane Associated Protein (APMAP), an agonist of fatty-acid G-protein coupled receptor GPR84, or a combination thereof. The methods may further comprise contacting the cell with at least one or both of a tumor antigen (TA)-targeting antibody and a CD47 blocking antibody. The present disclosure also provides methods for determining cellular regulators of phagocytosis.

### CROSS-REFERENCE TO RELATED APPLICATIONS

This application claims the benefit of U.S. Provisional Application No. 63/087,625, filed Oct. 5, 2020, the contents of which is herein incorporated by reference in their entirety.

### STATEMENT REGARDING FEDERAL FUNDING

This invention was made with government support under contract HD084069 awarded by the National Institutes of Health. The government has certain rights in the invention.

### FIELD

The present disclosure provides methods for treating a disease or disorder (e.g., cancer and autoimmune disorders), sensitizing cells to phagocytosis, and determining cellular regulators of phagocytosis.

### BACKGROUND

Macrophages, which are a type of white blood cells of the mononuclear phagocyte immune system, play vitally important roles in anti-infective immunity, the maintenance of tissue homeostasis, and the protection of a body through the functions of engulfing foreign substances through phagocytosis facilitating their breakdown and digestion. Macrophages also clear away harmful matter, including cellular debris and tumor cells in vivo. While monoclonal antibodies and CD47 blockade have been used as anti-cancer agents, in part by driving phagocytosis of tumor cells, existing therapies suffer from low response rates in patients.

### SUMMARY

Disclosed herein are methods of treating a disease or disorder or sensitizing a cell to phagocytosis comprising contacting a cell with an inhibitor of an anti-phagocytic gene or gene product. The anti-phagocytic gene or gene product may be selected from the group consisting of: GFI1; SMAGP; MUC21; ST6GALNAC1; OSR2; MUC1; CD47; GNE; GAL3ST4; ST3GAL1; CMAS; LRRC15; TLE3; PRDM1; SPN; MUC12; PTPRC; HDAC9; NFIA; NANS; GFI1B; POU2F2; IRX5; C1GALT1C1; QPCTL; SLC35A1; C5AR1; CD44; JMJD1C; CAB39; UBE2D3; PODXL, HMHA1; HIC1; PDCD10; SLA; C1GALT1; POU2AF1; PTEN; ZEB2; APMAP; SASH3; HES7; BCOR; PTPN6; RTN4IP1; RAC2; FOXO4; CAPN6; ST3GAL2; AIFM1; FDX1; CEBPE; NDUFA1; GTPBP6; FCGR1B; NDUFS8; TACO1; GPR114; CMC1; GRHL1; PNMA5; ATP5SL; SLC39A9; SLA2; ZBTB7A; CHMP1A; GRSF1; CD79B; ZNF683; CIITA; ZBTB7B; BCL6; C17ORF89; NDUFAF7, PDE12; MAK; UQCC1, MAP3K10; NDUFAF5; HIGD2A; TMEM119; SIX4; NDUFB9; GYPA; ZFX; MECR; RNF122; MED13; DBR1; MUC22, PWWP2B; WDR1; COX18; DBN1; TTC39C; NRG4; MSS51; NDUFS6; FOXO1; TMEM261; VSIG1; SORD; DGKB; C15ORF59; S100A11; CS; ADAD2; MS4A8; TRIM13; POU2F2; ADAM10; NDUFB6; NOMO2; NOMO3; PNMAL1; DOCK10; KCNS2; NOMO1; ZMYND8; SLC30A7; KCNJ6; VPS39; ZNF680; QSER1; CHAF1A; UNC13D; IGLL1; TIMM23B; MTIF3; PIGR; MYH10; NANOS3; MTO1; LPPR1; TIMM23; UBR4; CD4; KRT23; ARRDC3; RAB44; NDUFB4; JARID2; KRT6A; LIPT2; GK5; MPZL1; HMHA1; TMPRSS5; YBEY; ZNF521; RDX; ARHGAP30; OBP2A; ALAD; PCBP4; NXT1; RPL5; PRKAR1A; OTUB1; NDUFV1; GSTM2; GTPBP3; AMPD2; FXYD5; NUBPL; NDUFS2; NDUFB11; PPT1; ZEB1; ADRBK2; LACTBL1; POLR2H; SAMD4B; ZBTB7A; BTBD19; TIMMDC1; TRIM33; CCNT1; STARD7; AP000721.4; MAP1B, C20ORF166; NFIA; SEMA4A; UBE2K; VPS37A; NDUFA9; TMOD1; CEACAM1; COX5B; NDUFA8; ESRP1; FBRS; CTRC; PDK3; PTPRC; ACTB; NDUFAF3; FGD3; HMBS; NDUFC1; GMIP; BHLHA15; TMEM38B; LYN; NDUFS7; B3GNT7; FEZ2; MRPS2; PRKCD; MYCBP2; FLI1; TBX22; VPS37C; STUB1; NDUFS1; SMS; MRPL24; AHR; LIPT1; NLRC3; SORCS1; SCYL1; CLCC1; RPP14; XRN1; ZP2; OXER1; LENG9; C1QBP; MRPL37; UBE2E2; TBC1D22A; NOP58; CR2; KCMF1; COQ9; IRF2; MXRA5; TOMM70A; NDUFAF6; PLEKHO2; HSD17B12; or combinations thereof.

The anti-phagocytic gene or gene product may be selected from the group consisting of: DOCK2; CAPRIN1; STAG2; GSK3A; CFLAR; RBM12; BCLAF1; ELAVL1; SSR4; FBXW7; LIAS; ARL1; SRSF10; PPIH; CCDC6; COPG1; FAM58A; EEF1A1; ST6GAL1; PPP1R2; USP7; CLASRP; PTAR1; CDK6; GMDS; HECTD1; MYC; RUVBL1; VTA1; VPS26A; ACTR1A; PCSK7; SEC23B; ZNF281; ARPC3; PAG1; ATP5C1; PHACTR4; C19ORF43; SYMPK; SZRD1; MEF2BNB-MEF2B; RPP21; SDHC; INTS5; ARID2; COA3; PARS2; PTPMT1; COPB2; DDX55; TRA2A; VPS72; SF3B14; DUX4L5; RRP9; MRPS2; LIN54; OXSM; NDUFA6; NDUFB7; CIT; SUV420H1; NDUFB8; PSMD3; RP11-234B24.6; NPDC1; PDSS2; NAPA; NIPBL; EIF3B, PEPD; COX20; MYB; C1ORF233, RRAGC; SHQ1, UBE3D; NDUFA2; IER5L; SPPL3; NDUFS5; IKZF3; UBE2J2; PPOX; IDH3B; CYTH1; NDUFB10; TMEM9B; WDR26; YPEL5; ZBTB16; PTOV1; or combinations thereof.

In some embodiments, the anti-phagocytic gene or gene product comprises GFI1; SMAGP; MUC21; ST6GALNAC1; MUC1; GAL3ST4; LRRC15; MUC12; C5AR1; APMAP; or a combination thereof.

Disclosed herein are methods of treating a disease or disorder or sensitizing a cell to phagocytosis comprising contacting a cell with an inhibitor of Adipocyte Plasma Membrane Associated Protein (APMAP), an agonist of fatty-acid G-protein coupled receptor GPR84, or a combination thereof. In some embodiments, contacting the cell may comprise administration to a subject in need thereof (e.g., a subject having or expected of having cancer).

The agonist of fatty-acid G-protein coupled receptor GPR84 may comprise any known GPR84 agonist, including for example, lipids or synthetic agonists. The GPR84 agonist may comprise: medium chain fatty acid capric acid, ZQ-16, (octylamino) pyrimidine-2,4(1H,3H)-dione (6-n-octylaminouracil), 6-OAU), DL-175 (ACS Chem. Biol. 2019, 14, 9, 2055-2064), Diindolemethane derivatives (J. Med Chem. 2017, 60, 9, 3636-3655), 2-Alkylpyrimidine-4,6-diol, 6-Alkylpyridine-2,4-diol (ACS Med. Chem. Lett. 2016, 7, 6, 579-583), embelin (patent WO2007027661A2), or other known GPR84 agonists (see, e.g., J. Med. Chem. 2020, 63, 5, 2391-2410 and ACS Omega 2018, 3, 3, 3365-3383, each incorporated herein by reference in their entirety). In some embodiments the GPR84 agonist comprises medium chain fatty acid capric acid. In some embodiments the GPR84 agonist is selected from the group consisting of ZQ-16, (octylamino) pyrimidine-2,4(1H,3H)-dione (6-n-octylaminouracil, 6-OAU), or a combination thereof.

The methods may further comprise contacting the cell with at least one or both of a tumor antigen (TA)-targeting antibody and a CD47 blocking antibody. Any known TA-targeting or CD47 blocking antibody or blocking agent may be compatible with the disclosed methods, including, for example, anti-EGFR agents (e.g., cetuximab), anti-CD30 agents (e.g., brentuximab), an anti-CD47 antibody, an anti-SIRPalpha antibody, soluble SIRPa fragments, CD20 antibodies (e.g., rituximab, obinutuzumab, ofatumumab), and the like. In some embodiments the TA-targeting antibody comprises rituximab, cetuximab, brentuximab, or a combination thereof. In some embodiments, the CD47 blocking antibody comprises an anti-CD47 antibody, an anti-SIRPalpha antibody, or any combination thereof.

The methods may further comprise contacting the cell with an inhibitor an anti-phagocytic factor. In some embodiments, the anti-phagocytic factor is selected from the group consisting of PD-L1, CD24, or combinations thereof.

The disease or disorder may comprise any disease or disorder in which cell elimination by monoclonal antibodies or CD47 blockade or restoration of phagocytosis is the desired outcome, including, but not limited to: an autoimmune disorder, atherosclerosis, or cancer. In some embodiments, the disease or disorder is an autoimmune disorder, e.g., rheumatoid arthritis and multiple sclerosis.

In some embodiments, the disease or disorder is cancer. In some embodiments the cell is a cancer cell. The cancer or cancer cell may be any cancer. In some embodiments, the cancer or cancer cell is resistant to antibody-dependent cellular phagocytosis (ADCP). In some embodiments, the cancer or cancer cell overexpresses CD47. In some embodiments, the cancer or cancer cell is a solid tumor. In some embodiments, the cancer or cancer cell comprises lymphoma, cervical cancer, lung cancer (e.g., non-small-cell lung cancer and small-cell lung cancer), colorectal cancer, ovarian cancer, breast cancer and/or leukemia.

The disclosure also provides methods for identifying regulators of phagocytosis. In some embodiments the methods identify regulators of antibody-dependent cellular phagocytosis (ADCP) in target cells. The methods may comprise a) incubating cells with LPS-treated macrophages in the presences of anti-CD20, anti-EGFR, anti-CD30, and/or anti-CD47 antibodies, wherein the cells comprise a CRISPR knockout system or a CRISPR activation (CRISPRa) system and each cell comprises at least one guide RNA targeting an endogenous gene; b) separating unphagocytosed cells from macrophages; c) extracting nucleic acids from the unphagocytosed cells; and d) identifying the guide RNA and guide RNA endogenous gene targets in the unphagocytosed cells. In some embodiments, steps a) and b) are repeated at least once prior to step c). In some embodiments, the LPS-treated macrophages are treated with 10 ng/mL LPS 24 hours prior to incubation with the cells.

In some embodiments, the cell are cancer cells. In some embodiments, the cells are lymphoma cells (e.g., Ramos or Karpas-299 lymphoma cells). In some embodiments, the macrophages are J774 macrophage cells.

In some embodiments, the cells comprise a CRISPR knockout system and the incubation was in the presence of anti-CD20 antibodies. In alternate embodiments, the cells comprise a CRISPRa system and the incubation was in the presence of anti-CD20 and anti-CD47 antibodies.

In some embodiments, the identifying comprises sequencing the guide RNA.

In some embodiments, the methods identify regulators of phagocytosis in macrophages. The methods may comprise: a) incubating cells comprising a detectable label with LPS-treated macrophages, wherein the macrophages comprise a CRISPR knockout system or a CRISPR activation (CRISPRa) system and each macrophage comprises at least one guide RNA targeting an endogenous gene; b) removing unphagocytosed cells from macrophages; and c) separating the macrophages based on presence or absence of the detectable label. In some embodiments, the LPS-treated macrophages are treated with 10 ng/mL LPS 24 hours prior to incubation with the cells.

In some embodiments, the cell are cancer cells. In some embodiments, the cells are lymphoma cells (e.g., Ramos or Karpas-299 lymphoma cells). In some embodiments, the cells lack a regulator of phagocytosis (e.g., an endogenous gene determined by methods described herein as a regulator of phagocytosis). The cells may comprise any number of detectable labels (e.g., fluorescence, colorimetric, radioactive). In some embodiments, the detectable label is a fluorescent label. In some embodiments, the cells comprise more than one different types of cells each having a different fluorescent label. In some embodiments, the methods separate macrophages based on presence or absence of each individual detectable label. In some embodiments, the macrophages are J774 macrophage cells.

Other aspects and embodiments of the disclosure will be apparent in light of the following detailed description and accompanying figures.

### BRIEF DESCRIPTION OF FIGURES

FIGS. 1A-1G show that genome-wide CRISPR screens reveal novel regulators of antibody-dependent cancer cell phagocytosis. FIG. 1A is a schematic of genome-wide CRISPR knockout screen in Ramos cells for sensitivity to phagocytosis. FIG. 1B is a volcano plot of genome-wide screen in Ramos cells depicted in FIG. 1A. FIG. 1C is a volcano plot of genome-wide CRISPRa screen in Ramos cells depicted in FIG. 1A. FIG. 1D is a survival assay for Ramos cells expressing indicated sgRNAs in presence of J774 macrophages and anti-CD20 antibodies. GFP+ sgSafe-expressing cells were mixed with equal number of mCherry+ cells expressing indicated sgRNAs and cultured in presence of J774 macrophage and anti-CD20 antibodies. Mean percentage of cells that are mCherry-positive after 2 d, normalized to sgCtrl-1 cells, is indicated (n=3, error bars represent standard deviation). FIG. 1E is a graph of phagocytosis assay for uptake of pHrodo-labeled Ramos CRISPRa cells expressing indicated sgRNAs by J774 macrophages in the presence of anti-CD20 antibodies. Normalized phagocytosis index was calculated as average total pHrodo Red signal per well, normalized to signal in sgCtrl-1 cells at 10 h timepoint. Data represent mean+/−s.d. (n=4). FIG. 1F is graph of expression (TPM) of SMAGP in 1304 cell lines in CCLE. FIG. 1G is graph of phagocytosis assay for uptake of pHrodo-labeled RKO Cas9 cells expressing indicated sgRNAs by J774 macrophages in the presence or absence of anti-CD47 antibodies. Normalized phagocytosis index was calculated as average total pHrodo Red signal per well, normalized to signal in sgCtrl-1 cells at 10 h timepoint. Data represent mean+/−s.d. (n=4).

FIGS. 2A-2F show that APMAP deficiency synergizes with CD47 blockade in enhancing cancer cell sensitivity to phagocytosis. FIG. 2A is images of pHrodo-labeled GFP+ Karpas-299 cells expressing indicated sgRNAs after 0 h and 10 h incubation with J774 macrophages. Scale bar=50 μm. FIG. 2B is a graph of phagocytosis assay for uptake of pHrodo-labeled Karpas-299 cells with indicated genotypes by J774 macrophages in the presence or absence of anti-CD30 antibodies. FIG. 2C is a graph of phagocytosis assay for uptake of pHrodo-labeled Ramos cells with indicated genotypes by human U937 macrophages in the presence of anti-CD20 antibodies. FIG. 2D is a volcano plot of CRISPR screen in Ramos cells for sensitivity to macrophage phagocytosis in the presence of anti-CD20 in cells expressing an sgRNA targeting CD47. FIG. 2E is a graph of phagocytosis assay for uptake of pHrodo-labeled Ramos cells with indicated genotypes by J774 macrophages in the presence of anti-CD47 antibodies. FIG. 2F is a graph of phagocytosis assay for uptake of pHrodo-labeled Ramos cells expressing indicated sgRNAs, incubated for 6h with J774 macrophages in the presence of anti-CD20 and/or anti-CD47 antibodies. Normalized phagocytosis index was calculated as average total pHrodo Red signal per well, normalized to signal in Safe^{KO} cells. Data represent mean+/−s.d. (n=4).

FIGS. 3A-3D show that APMAP loss sensitizes diverse tumor types to ADCP in vitro and in mice. FIG. 3A is graphs of phagocytosis assay for uptake of pHrodo-labeled cells for indicated Cas9-expressing cell lines expressing indicated sgRNAs by J774 macrophages in the presence or absence of anti-CD47 antibodies. FIG. 3B is graphs of phagocytosis assay for uptake of pHrodo-labeled cells for indicated Cas9-expressing cell lines expressing indicated sgRNAs by J774 macrophages in the presence or absence of anti-EGFR/cetuximab antibodies. Safe^{KO} or APMAP^{KO} Ramos cells were transplanted into mice and allowed to form tumors. Mice were treated with anti-CD47 or PBS daily starting 17 d following transplantation, and tumor size was measured every 2d (FIG. 3C). Safe^{KO} or APMAP^{KO} NCI-H82 cells were transplanted into mice and allowed to form tumors. Mice were treated with anti-CD47 or PBS daily starting 12 d following transplantation, and tumor size was measured every 2d (FIG. 3D).

FIGS. 4A-4H show that GPR84 mediates enhanced uptake of APMAP^{KO} cancer cells. FIG. 4A is a schematic of macrophage screen, using 2,208-gene sublibrary (enriched for phagocytosis genes) in J774 macrophages, for uptake of calcein+ Safe^{KO} cells and far-red+ APMAP^{KO} Ramos cells. FIG. 4B is a volcano plot of screen diagrammed in FIG. 4A. FIG. 4C is graphs of phagocytosis assay for uptake of pHrodo-labeled Ramos cells expressing indicated sgRNAs, incubated with J774 macrophages expressing indicated sgRNAs, in the presence of anti-CD20 antibodies. Phagocytosis index (arbitrary units) corresponds to the total pHrodo Red signal per well. Data represent mean+/−s.d. (n=4). FIG. 4D is graphs of phagocytosis assay for uptake of pHrodo-labeled Ramos cells expressing indicated sgRNAs, incubated with J774 macrophages, in the presence of anti-CD20 antibodies, with or without addition of indicated GPR84 agonists at indicated concentrations. Phagocytosis index (arbitrary units) corresponds to the total pHrodo Red signal per well. Data represent mean+/−s.d. (n=4). FIG. 4E is a schematic of APMAP structure. FIG. 4F is a view of catalytic site of APMAP homology model. Ca, calcium. FIG. 4G is a graph of phagocytosis assay for uptake of pHrodo-labeled Ramos cells expressing indicated sgRNAs, incubated with J774 macrophages expressing indicated sgRNAs, in the presence of anti-CD20 antibodies. Phagocytosis index (arbitrary units) corresponds to the total pHrodo Red signal per well. Data represent mean+/−s.d. (n=4). FIG. 4H is a model for APMAP-mediated regulation of macrophage phagocytosis through GPR84.

FIG. 5A is a graph of replicates for CRISPRko screen in Ramos cells for susceptibility to ADCP driven by anti-CD20 antibodies. FIG. 5B is a graph of replicates for CRISPRa screen in Ramos cells for susceptibility to ADCP driven by anti-CD20 and anti-CD47 antibodies.

FIG. 6 is gene ontology enrichment analysis for 336 hits from CRISPR screens defined using CasTLE score threshold of 50 (150 negative CRISPRko hits, 195 positive CRISPRa hits; 9 genes were in both categories).

FIG. 7A is a graph of cell-cell adhesion assay for binding of GFP+ Ramos CRISPRa cells expressing indicated sgRNAs to J774 cells. J774 cells were treated with cytochalasin D to block phagocytosis, then incubated with Ramos cells for 1 h, washed 3 times with PBS, and imaged using an Incucyte. FIG. 7B is a Venn diagram of putative anti-phagocytic factors identified in CRISPR screens in FIGS. 1B and 1C (negative hits in CRISPRko screen and positive hits in CRISPRa screen).

FIG. 8 is a graph of phagocytosis assay for uptake of pHrodo-labeled Ramos Cas9 cells expressing indicated sgRNAs by J774 macrophages in the presence of indicated concentrations of anti-CD20 antibodies. Normalized phagocytosis index was calculated as average total pHrodo Red signal at 5 h for each well, normalized to signal in sgCtrl-1 cells at 5 h timepoint. Data represent mean+/−s.d. (n=4).

FIG. 9A is a graph of annexin V staining of Ramos cells expressing indicated sgRNAs.

FIG. 9B is a cell survival assay with equal mixture of GFP-expressing Safe^{KO} Ramos Cas9 cells and mCherry-expressing cells with indicated sgRNAs following treatment with indicated concentrations of hydrogen peroxide. FIG. 9C is a graph of surface levels of CD47 in Ramos cells expressing indicated sgRNAs.

FIG. 10A is a volcano plot of CRISPR screen in Ramos cells for sensitivity to macrophage phagocytosis in the presence of anti-CD20 in cells expressing an sgRNA targeting a Safe locus. FIG. 10B is a volcano plot of CRISPR screen in Ramos cells for sensitivity to macrophage phagocytosis in the presence of anti-CD20 and anti-CD47 in cells expressing an sgRNA targeting a Safe locus.

FIG. 11 is images of Ramos tumors of indicated genotype extracted from NSG mice at 25 d following transplantation.

FIG. 12A is a volcano plot for macrophage screen for genes required selectively for uptake of APMAP^{KO} cells, following screen design in FIG. 4A, but using 2,208-gene sublibrary (enriched for phagocytosis genes) in J774 macrophages, for uptake of calcein+ Safe^{KO} cells and far-red+ APMAP^{KO} Ramos cells. FIG. 12B is a FACS-based phagocytosis assay for uptake of CellTrace Far-Red labeled APMAP^{KO} cells and calcein-labeled Safe^{KO} cells by J774 Cas9 macrophages expressing indicated sgRNAs. FIG. 12C is graphs of phagocytosis assay for uptake of pHrodo-labeled Ramos Cas9 cells expressing APMAP targeting sgRNAs by J774 macrophages in the presence of anti-CD20 antibodies and indicated concentrations of GPR84 agonists.

FIG. 13A is images of localization of APMAP-FLAG and APMAP^{E103}A-FLAG to the endoplasmic reticulum in HeLa cells. FIG. 13B is APMAP^{KO} Ramos cells transduced with indicated constructs were subjected to labeling with EZ-NHS-SS-Biotin (NHS-Biotin), lysed, and incubated with streptavidin magnetic beads to isolate cell surface proteins. Biotinylated proteins were eluted with DTT, separated by SDS-PAGE, and analyzed by immunoblotting with indicated antibodies. FIG. 13C is immunoblotting of cell extracts that were treated, where indicated, with PNGase F to remove N-glycosylation. FIG. 13D is a graph of phagocytosis assay for uptake of pHrodo-labeled Ramos Cas9 cells expressing indicated sgRNAs and indicated addback constructs by J774 macrophages in the presence of anti-CD20 antibodies. Data represent mean+/−s.d. (n=4). FIG. 13E is a graph of phagocytosis assay for uptake of pHrodo-labeled Ramos Cas9 cells expressing indicated sgRNAs and indicated addback constructs by J774 macrophages in the presence of anti-CD20 antibodies. Data represent mean+/−s.d. (n=4).

FIGS. 14A-14F show that genome-wide CRISPR screens reveal novel regulators of ADCP. FIG. 14A is a volcano plot of genome-wide screen in Ramos cells. FIG. 14B is a volcano plot of a genome-wide CRISPRa screen in Ramos cells. FIG. 14C is a diagram of top 50 anti-phagocytic hits from CRISPRko (pink) and CRISPRa (blue) screens in Ramos cells. SIRPA, the macrophage receptor for CD47, is in gray. FIG. 14D is a graph of phagocytosis assay for uptake of pHrodo-Red-labeled Ramos CRISPRa cells expressing indicated sgRNAs by J774 macrophages in the presence of anti-CD20 and anti-CD47 antibodies. Phagocytosis index was calculated as total pHrodo Red signal (which increases in the low-pH environment of the lysosome) per well, normalized to signal in cells expressing a Safe-targeting control sgRNA (5 h timepoint) (n=3 cell culture wells). FIG. 14E is a schematic of target cell-macrophage adhesion assay, left, and a graph of the proportion of Ramos cells expressing indicated sgRNAs bound to macrophages is plotted, normalized to Ramos cells expressing Safe-targeting control sgRNA (n=3 cell culture wells), right. FIG. 14F is a comparison of Ramos/anti-CD20 and Karpas-299/anti-CD30 ADCP screens.

FIGS. 15A-15D show APMAP loss synergizes with mAbs and CD47 blockade to increase cancer cell susceptibility to phagocytosis. FIG. 15A is images of pHrodo-labeled GFP⁺ Ramos cells of indicated genotypes after 12 h incubation with J774 macrophages with or without anti-CD20. Scale bar=50 μm. Representative of 3 biologically independent experiments. FIG. 15B is a volcano plot of genome-wide CRISPRko screen in Karpas-299-Cas9 cells for resistance to treatment with anti-CD47 and macrophages. Dotted line indicates 5% FDR. FIG. 15C is a graph of a phagocytosis assay for uptake of pHrodo-labeled Ramos cells with indicated genotypes following incubation with J774 macrophages and anti-CD47 antibodies or isotype control (mIgG1) antibodies, normalized to control (Safe^{KO}) cells with isotype control antibody (n=3 cell culture wells). FIG. 15D is a graph of a phagocytosis assay for uptake of pHrodo-labeled Ramos cells expressing indicated sgRNAs, incubated for 2 h with J774 macrophages and anti-CD20, anti-CD47, or indicated isotype control antibodies, normalized to signal in control (SafeKO) cells in anti-CD20/mIgG1-isotype condition (n=3 cell culture wells).

FIGS. 16A-16C show GPR84 mediates enhanced uptake of APMAP^{KO} cancer cells. FIG. 16A is a volcano plot of all genes required for uptake of APMAP^{KO} cells. FIG. 16B is a graph of a phagocytosis assay for uptake of pHrodo-labeled Ramos-Cas9 cells expressing indicated sgRNAs, incubated with J774 macrophages expressing indicated sgRNAs, in the presence of anti-CD20 antibodies (n=4 cell culture wells). FIG. 16C is a graph of a phagocytosis assay for uptake of pHrodo-labeled Safe^{KO} Ramos cells expressing indicated sgRNAs, incubated with U937 macrophages, anti-CD20 antibodies and indicated GPR84 agonists at indicated concentrations. Phagocytosis index (arbitrary units) corresponds to the total pHrodo Red signal per well normalized to untreated cells (n=4 cell culture wells). P-values are for comparisons to untreated cells.

FIG. 17 is a graph of phagocytosis assay for uptake of pHrodo-labeled Ramos-Cas9 cells with indicated genotypes by J774 macrophages in the presence of anti-CD20 antibodies. APMAP-cytosolic truncation. Phagocytosis index normalized to control (Safe^{KO}) cells. Data represent mean+/−s.d. (n=4 cell culture wells). One-way ANOVA with Bonferroni correction.

FIGS. 18A-18L show evaluation of the role of APMAP in ADCP across diverse cancer cell lines and in syngeneic mice. FIG. 18A shows levels of APMAP in ten cell lines measured by Western blot. All cell lines stably express Cas9 and were transduced with indicated sgRNAs. Actin served as loading control. Western blot to confirm knockout across all ten cell lines on one gel was performed once. FIG. 18B is a graph of expression levels (TPM) of CD47 and APMAP in ten cell lines (data from CCLE). FIG. 18C is graphs of survival measurements of selected (GFP⁺) cell lines, measured as percentage of GFP remaining after indicated number of hours of incubation with J774 macrophages in presence or absence of anti-CD47. Data represent mean+/−s.d. (n=4 cell culture wells, except Karpas-299 (n=3)). One-way ANOVA with multiple comparisons correction. FIG. 18D is graphs of phagocytosis assays, but with isotype control antibodies. Data represent mean+/−s.d. (n=4 cell culture wells). One-way ANOVA with Bonferroni correction. FIG. 18E is graphs of survival measurements of selected (GFP*) cell lines, measured as percentage of GFP remaining after indicated number of hours of incubation with J774 macrophages in presence or absence of anti-EGFR. Data represent mean+/−s.d. (n=3 cell culture wells). One-way ANOVA with Bonferroni correction. FIG. 18F shows Safe^{KO} or APMAP^{KO} Ramos cells were transplanted into NSG mice and allowed to form tumours. Mice were treated with anti-CD20 or PBS daily starting 21 d following transplantation, and tumour size was measured every 2 d. Data represent mean+/−s.e.m. (n=6 (control groups) and 7 (antibody-treated groups)). Two-way ANOVA with Tukey correction. FIG. 18G is graphs of mouse weights in Ramos (top) and NCI-H82 (bottom) xenograft experiments. Data represent mean+/−s.d. Two-way ANOVA with Bonferroni correction (n=5 (all NCI-H82 groups and Ramos Safe^{KO} groups) and 6 (Ramos APMAP^{KO} groups)). P-values are reported for the interaction between treatment groups. FIG. 18H is single-cell suspensions were prepared from Safe^{KO} or APMAP^{KO} Ramos tumors treated with PBS or anti-CD20 and analyzed for the presence of macrophages (CD45⁺/F4-80⁺/Cd11b⁺) as a percentage of all CD45⁺ cells. Gating strategy is shown (top/left). Data (bottom right) represent mean+/−s.e.m (n=6 (PBS groups) and 7 (antibody-treated groups)). One-way ANOVA with Tukey correction. FIG. 18I is a graph of a phagocytosis assay for uptake of pHrodo-labeled B16-F10 cells with indicated genotypes by J774 macrophages in the presence or absence of anti-TRP1 antibodies. Phagocytosis index normalized to control (Safe^{KO}) cells without antibody. Data represent mean+/−s.d. (n=4 cell culture wells). One-way ANOVA with Bonferroni correction. FIG. 18J is a graph of in vitro growth of B16-F10 cells of indicated genotypes, measured using time-lapse microscopy as total confluence per well over 6 d. Data represent mean+/−s.d. (n=4 cell culture wells). FIG. 18K shows Safe^{KO} or APMAP^{KO} B16-F10 cells were transplanted into syngeneic C57BL/6 mice and allowed to form tumors. Mice were treated with anti-TRP1 or mouse IgG2a isotype control antibody daily starting 5 d following transplantation, and tumor size was measured every 2 d. Data represent mean+/−s.e.m (n=7 for Safe^{KO} groups, n=6 for both APMAP^{KO} groups). Two-way ANOVA with Tukey correction (comparison between Safe^{KO}/anti-TRP1 and APMAP^{KO}/anti-TRP1 for final timepoint is shown).

FIG. 19A-19E show genome-wide magnetic screen in J774 macrophages for phagocytosis of IgG-coated beads. FIG. 19A is a schematic of genome-wide screen in J774 macrophages for phagocytosis of 2.8 micron IgG-coated magnetic beads. FIG. 19B is a volcano plot of screen diagrammed in FIG. 19A. Dotted line indicates 5% FDR. FIG. 19C is replicates of the screen diagrammed in FIG. 19A. FIG. 19D is a diagram of hits with negative effect size (e.g., required for phagocytosis) from genome-wide screen for IgG bead phagocytosis in J774 macrophages. FIG. 19E is gene ontology enrichment analysis for macrophage IgG bead screen hits with negative effect size (required for phagocytosis) (5% FDR). Selected terms shown. n indicates number of genes among hits annotated with indicated term.

### DETAILED DESCRIPTION

The disclosed compositions, systems, and methods allowed unbiased identification of anti-phagocytic factors using genome-wide CRISPR screens. In addition to CD47, several novel regulators of cancer cell susceptibility to antibody-dependent cellular phagocytosis (ADCP), including the poorly characterized cell surface enzyme APMAP (Adipocyte Plasma Membrane Associated Protein), were identified. Loss of APMAP synergized with tumor antigen (TA)-targeting mAbs and/or CD47 blocking mAbs to drive dramatically increased rates of phagocytosis across a wide range of cancer cell types, including those that are otherwise resistant to ADCP. Additionally, APMAP loss enhanced control of tumor development in mice in a synergistic manner with CD47-blocking mAbs. Using genome-wide CRISPR screens in macrophages, a signaling pathway, comprising the fatty-acid G-protein coupled receptor GPR84, the G-protein GNB2, and the downstream actin regulator PREX1, that is required for enhanced phagocytosis of APMAP-deficient cancer cells was identified, although an understanding of the mechanism of action is not required to practice the invention. This analysis revealed a novel targetable cancer liability that specifically sensitized mAb-bound cancer cells to killing by macrophages and demonstrated the ability of genome-wide CRISPR screens to uncover novel intercellular signaling pathways that regulate cancer immunotherapy. Identified regulators of phagocytosis are listed in Table 1 and Table 2.

### 1. DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The term “agonist,” as used herein, refers to a substance (e.g., small molecule, protein, and the like) that mimics or has the same function as a natural binding ligand or partner. For example, a receptor agonist is a substance that binds a receptor and causes the same action as the natural or endogenous binding ligand or partner.

The term “antagonist,” as used herein, refers to a substance that interferes with or inhibits the natural action or function of a cellular constituent.

“Polynucleotide” or “oligonucleotide” or “nucleic acid,” as used herein, means at least two nucleotides covalently linked together. The polynucleotide may be DNA, both genomic and cDNA, RNA, or a hybrid, where the polynucleotide may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. Polynucleotides may be single- or double-stranded or may contain portions of both double stranded and single stranded sequence. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The proteins may be modified by the addition of sugars, lipids or other moieties not included in the amino acid chain. The terms “polypeptide” and “protein” are used interchangeably herein.

As used herein, “treat,” “treating” and the like mean a slowing, stopping, or reversing of progression of a disease or disorder when provided a composition described herein to an appropriate control subject. The term also means a reversing of the progression of such a disease or disorder to a point of eliminating or greatly reducing the cell proliferation. As such, “treating” means an application or administration of the methods described herein to a subject, where the subject has a disease or a symptom of a disease, where the purpose is to cure, heal, alleviate, relieve, alter, remedy, ameliorate, improve, or affect the disease or symptoms of the disease.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods and compositions provided herein, the mammal is a human.

The term “contacting” as used herein refers to bring or put in contact, to be in or come into contact. The term “contact” as used herein refers to a state or condition of touching or of immediate or local proximity. Contacting inhibitors, antagonists, and agonists of the disclosed methods to a target destination, such as, but not limited to, an organ, tissue, cell, or tumor, may occur by any means of administration known to the skilled artisan.

As used herein, the terms “providing,” “administering,” “introducing,” are used interchangeably herein and refer to the placement into a subject by a method or route which results in at least partial localization to a desired site. The inhibitors, antagonists and agonists of the disclosed methods can be administered by any appropriate route which results in delivery to a desired location in the subject.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. SENSITIZING A CELL TO PHAGOCYTOSIS

The present disclosure provides methods comprising contacting a cell with an inhibitor of an anti-phagocytic gene or gene product. Phagocytosis is a basic process for nutrition in unicellular organisms and is found in almost all cell types of multicellular organisms. A specialized group of cells (e.g., macrophages, neutrophils, monocytes, dendritic cells, osteoclasts) accomplish phagocytosis with high efficiency and are primarily responsible for the removal of microorganisms and presentation of antigens to lymphocytes as part of the adaptive immune response. Other cell types can also participate in more general uses for phagocytosis including eliminating dead cells and maintaining homeostasis. Anti-phagocytic genes include those that prevent or negatively control phagocytosis.

In some embodiments, the anti-phagocytic genes may be expressed in certain cell or tissue types. For example, in some embodiments, an anti-phagocytic gene may be expressed in a diseased cell (e.g., a cancer cell) but not in normal cells.

In some embodiments, the anti-phagocytic gene or gene product is selected from the group consisting of: GFI1; SMAGP; MUC21; ST6GALNAC1; OSR2; MUC1; CD47; GNE; GAL3ST4; ST3GAL1; CMAS; LRRC15; TLE3; PRDM1; SPN; MUC12; PTPRC; HDAC9; NFIA; NANS; GFI1B; POU2F2; IRX5; C1GALT1C1; QPCTL; SLC35A1; C5AR1, CD44; JMJD1C; CAB39; UBE2D3; PODXL; HMHA1; HIC1; PDCD10; SLA; C1GALT1; POU2AF1; PTEN; ZEB2; APMAP; SASH3; HES7; BCOR; PTPN6; RTN4IP1; RAC2; FOXO4; CAPN6; ST3GAL2; AIFM1; FDX1; CEBPE; NDUFA1; GTPBP6; FCGR1B; NDUFS8; TACO1; GPR114; CMC1, GRHL1; PNMA5; ATP5SL; SLC39A9; SLA2; ZBTB7A; CHMP1A; GRSF1; CD79B; ZNF683; CIITA; ZBTB7B; BCL6; C17ORF89; NDUFAF7; PDE12; MAK; UQCC1; MAP3K10; NDUFAF5; HIGD2A; TMEM119; SIX4; NDUFB9; GYPA; ZFX; MECR; RNF122; MED13; DBR1; MUC22; PWWP2B; WDR1; COX18; DBN1; TTC39C; NRG4; MSS51; NDUFS6; FOXO1; TMEM261; VSIG1; SORD; DGKB; C15ORF59; S100A11; CS; ADAD2; MS4A8; TRIM13; POU2F2; ADAM10; NDUFB6; NOMO2; NOMO3; PNMAL1; DOCK10; KCNS2; NOMO1; ZMYND8; SLC30A7; KCNJ6; VPS39; ZNF680; QSER1; CHAF1A; UNC13D; IGLL1; TIMM23B; MTIF3; PIGR; MYH10; NANOS3; MTO1; LPPR1; TIMM23; UBR4; CD4; KRT23; ARRDC3; RAB44; NDUFB4; JARID2; KRT6A; LIPT2; GK5; MPZL1; HMHA1; TMPRSS5; YBEY, ZNF521; RDX, ARHGAP30; OBP2A; ALAD; PCBP4; NXT1; RPL5; PRKAR1A; OTUB1; NDUFV1; GSTM2; GTPBP3; AMPD2; FXYD5; NUBPL; NDUFS2; NDUFB11; PPT1; ZEB1; ADRBK2; LACTBL1; POLR2H; SAMD4B; ZBTB7A; BTBD19; TIMMDC1; TRIM33; CCNT1; STARD7; AP000721.4; MAP1B; C20ORF166; NFIA; SEMA4A; UBE2K; VPS37A; NDUFA9; TMOD1; CEACAM1; COX5B; NDUFA8; ESRP1; FBRS; CTRC; PDK3; PTPRC; ACTB; NDUFAF3; FGD3; HMBS; NDUFC1; GMIP; BHLHA15; TMEM38B; LYN; NDUFS7; B3GNT7; FEZ2; MRPS2; PRKCD; MYCBP2; FLI1; TBX22; VPS37C; STUB1; NDUFS1; SMS; MRPL24; AHR; LIPT1; NLRC3; SORCS1; SCYL1; CLCC1; RPP14; XRN1; ZP2; OXER1; LENG9; C1QBP; MRPL37; UBE2E2; TBC1D22A; NOP58; CR2; KCMF1; COQ9; IRF2; MXRA5; TOMM70A, NDUFAF6; PLEKHO2; HSD17B12; or combinations thereof.

In some embodiments, the anti-phagocytic gene or gene product is selected from the group consisting of: DOCK2; CAPRIN1; STAG2; GSK3A; CFLAR; RBM12; BCLAF1; ELAVL1; SSR4; FBXW7; LIAS; ARL1; SRSF10; PPIH; CCDC6; COPG1; FAM58A; EEF1A1; ST6GAL1; PPP1R2; USP7; CLASRP; PTAR1; CDK6; GMDS; HECTD1; MYC; RUVBL1; VTA1; VPS26A; ACTR1A; PCSK7; SEC23B; ZNF281; ARPC3; PAG1; ATP5C1; PHACTR4; C19ORF43; SYMPK; SZRD1; MEF2BNB-MEF2B; RPP21; SDHC; INTS5; ARID2; COA3; PARS2; PTPMT1; COPB2; DDX55; TRA2A; VPS72; SF3B14; DUX4L5; RRP9; MRPS2; LIN54; OXSM; NDUFA6; NDUFB7; CIT; SUV420H1; NDUFB8; PSMD3; RP11-234B24.6; NPDC1; PDSS2; NAPA; NIPBL; EIF3B; PEPD; COX20; MYB; C1ORF233; RRAGC; SHQ1; UBE3D; NDUFA2; IER5L; SPPL3; NDUFS5; IKZF3; UBE2J2; PPOX; IDH3B; CYTH1; NDUFB10; TMEM9B; WDR26; YPEL5; ZBTB16; PTOV1; or combinations thereof.

In select embodiments, the anti-phagocytic gene or gene product comprises GFI1; SMAGP; MUC21; ST6GALNAC1; MUC1; GAL3ST4; LRRC15; MUC12; C5AR1; APMAP; or a combination thereof.

Also provided are methods comprising contacting a cell with an inhibitor of Adipocyte Plasma Membrane Associated Protein (APMAP), an antagonist of Small Cell Adhesion Glycoprotein (SMAGP), an agonist of fatty-acid G-protein coupled receptor GPR84, or a combination thereof.

The inhibitors, agonists, or antagonists may be any substance (e.g., nucleic acid, proteins, polysaccharides, nucleotides, amino acids, monosaccharides or simple sugars, small molecules) which modulates (e.g., activates or inhibits) with the transcription, translation or action of the disclosed anti-phagocytic gene, Adipocyte Plasma Membrane Associated Protein (APMAP), an antagonist of Small Cell Adhesion Glycoprotein (SMAGP), an agonist of fatty-acid G-protein coupled receptor GPR84, in a specific manner. In some embodiments, the inhibitors, agonists, or antagonists include nucleic acid based substances and systems which modulate transcription or translation, including but not limited to, small interfering RNA or CRISRPR knockout systems. In some embodiments, the inhibitors, agonists, or antagonists include substances and systems which modulate the action of the gene product, including but not limited to, antibodies and small molecule inhibitors. Exemplary inhibitors, agonists, or antagonists are known in the art. Table 3 includes exemplary inhibitors, agonists, or antagonists for select anti-phagocytic genes or gene products.

G protein-coupled receptor 84 (GPR84) is a free fatty acid receptor activated by medium-chain free fatty acids with 9-14 carbons. In some embodiments, agonists of fatty-acid G-protein coupled receptor GPR84 comprise a lipid or a synthetic agonist. In some embodiments, the agonist of fatty-acid G-protein coupled receptor GPR84 comprises a medium chain fatty acid. In some embodiments, the medium chain fatty acid comprises capric acid. In some embodiments the agonist of fatty-acid G-protein coupled receptor GPR84 is selected from the group consisting of ZQ-16, (octylamino) pyrimidine-2,4(1H,3H)-dione (6-n-octylaminouracil, 6-OAU), or a combination thereof. Other GRP84 agonists are known in the in art including, but not limited to, those in U.S. patent application Ser. No. 15/772,105.

In some embodiments, the methods further comprise contacting the cell with an inhibitor of an anti-phagocytic gene or factor. Anti-phagocytic genes and factors include those that prevent or negatively control phagocytosis and may include any genes or factors which include that functionality or those described as elsewhere herein. In some embodiments, the anti-phagocytic factor is selected from the group consisting of PD-L1, CD24, or combinations thereof.

In some embodiments, the methods may be used to treat a disease or disorder. The disease or disorder may comprise an autoimmune disorder, cancer, or atherosclerosis.

In some embodiments, the disease or disorder is cancer. In some embodiments, the cancer comprises a solid tumor. In some embodiments, the cancer is metastatic cancer. In some embodiments, the disclosed methods result in suppression of elimination of metastasis. In some embodiments, the disclosed methods result in decreased tumor growth. In some embodiments, the disclosed methods prevent tumor recurrence. In instances when the disease or disorder is cancer, the cell being contacted may be a cancer cell. In some embodiments, the cancer or cancer cell is resistant to antibody-dependent cellular phagocytosis (ADCP). In some embodiments, the cancer or cancer cell overexpresses CD47.

The disclosed methods may be useful to treat a wide variety of cancers including carcinoma, sarcoma, lymphoma, leukemia, melanoma, mesothelioma, multiple myeloma, or seminoma. The cancer may be a cancer of the bladder, blood, bone, brain, breast, cervix, colon/rectum, endometrium, head and neck, kidney, liver, lung, lymph nodes, muscle tissue, ovary, pancreas, prostate, skin, spleen, stomach, testicle, thyroid, or uterus. In select embodiments, the cancer comprises lymphoma, cervical cancer, lung cancer, colorectal cancer, ovarian cancer, breast cancer and/or leukemia.

In some embodiments, the disease or disorder is an autoimmune disorder. Autoimmune diseases and disorders refer to conditions in a subject characterized by cellular, tissue and/or organ injury caused by an immunologic reaction of the subject to its own cells, tissues and/or organs. Autoimmune diseases and disorders that may be treated by the methods of the present invention include, but are not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), irritable bowel disease (IBD), IgA neuropathy, juvenile arthritis, lichen planus, lupus erythematosus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychondritis, polyglandular syndromes, polymyalgia rheumatics, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, takayasu arteritis, temporal arteritis/giant cell arteritis, ulcerative colitis, uveitis, vasculitides such as dermatitis herpetiformis vasculitis, vitiligo, and Wegener's granulomatosis. In select embodiments, the disease or disorder comprises rheumatoid arthritis or multiple sclerosis.

In some embodiments, the disease or disorder is atherosclerosis. Atherosclerosis comprises any disease or disorder characterized by the deposition of fats, cholesterol, and other substances in and on the walls of an artery causing the arteries to narrow thereby blocking blood flow or leading to a blood clot. Atherosclerosis and atherosclerotic associated diseases can affect arteries anywhere in your body and include but are not limited to coronary heart disease, carotid artery disease, chronic kidney disease and peripheral arterial disease.

The method may comprise administering to a subject, in vivo an effective amount of the described inhibitors, agonists, or antagonists. In some embodiments, the described inhibitors, agonists, or antagonists are delivered to a tissue of interest by, for example, an intramuscular, intravenous, transdermal, intranasal, oral, mucosal, or other delivery methods.

When utilized as a method of treatment, the effective amount may depend on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human.

The described inhibitors, agonists, or antagonists may be administered as a composition which further comprises a pharmaceutically acceptable carrier. The phrase “pharmaceutically acceptable,” as used in connection with compositions and/or cells of the present disclosure, refers to molecular entities and other ingredients of such compositions that are physiologically tolerable and do not typically produce untoward reactions when administered to a subject (e.g., a mammal, a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions and/or cells to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g., Remington: The Science and Practice of Pharmacy 20^{th} Ed. (2000) Lippincott Williams and Wilkins, Ed. K. E. Hoover.

A wide range of second therapies may be used with the disclosed methods. The second therapy may be administration of a therapeutic agent or may be a second therapy not connected to administration of another agent. Such second therapies include, but are not limited to, surgery, immunotherapy, radiotherapy, a chemotherapeutic or anti-cancer agent, a statin or other cholesterol controlling medication, a blood thinner, and blood pressure medications. As used herein, the term “chemotherapeutic” or “anti-cancer agent” includes any small molecule or other drug used in cancer treatment or prevention. Chemotherapeutics include, but are not limited to, cyclophosphamide, methotrexate, 5-fluorouracil, doxorubicin, docetaxel, daunorubicin, bleomycin, vinblastine, dacarbazine, cisplatin, paclitaxel, raloxifene hydrochloride, tamoxifen citrate, abemacicilib, afinitor, alpelisib, anastrozole, pamidronate, anastrozole, exemestane, capecitabine, epirubicin hydrochloride, eribulin mesylate, toremifene, fulvestrant, letrozole, gemcitabine, goserelin, ixabepilone, emtansine, lapatinib, olaparib, megestrol, neratinib, palbociclib, ribociclib, talazoparib, thiotepa, toremifene, methotrexate, and tucatinib.

The second therapy (e.g., an immunotherapy) may be administered at the same time as the disclosed methods, either in the same composition or in a separate composition administered at substantially the same time. In some embodiments, the second therapy may precede or follow the disclosed methods by time intervals ranging from hours to months.

In some embodiments, the second therapy includes immunotherapy. Immunotherapies include chimeric antigen receptor (CAR) T-cell or T-cell transfer therapies, cytokine therapy, immunomodulators, cancer vaccines, or administration of antibodies (e.g., monoclonal antibodies).

In some embodiments, the immunotherapy comprises administration of antibodies. The antibodies may target antigens either specifically expressed by tumor cells or antigens shared with normal cell. In some embodiments, the immunotherapy may comprise an antibody targeting, for example, CD20, CD33, CD52, CD30, HER (also referred to as erbB or EGFR), VEGF, CTLA-4 (also referred to as CD152), epithelial cell adhesion molecule (EpCAM, also referred to as CD326), and PD-1/PD-L1. Suitable antibodies include, but are not limited to, rituximab, blinatumomab, trastuzumab, gemtuzumab, alemtuzumab, ibritumomab, tositumomab, bevacizumab, cetuximab, panitumumab, ofatumumab, ipilimumab, brentuximab, pertuzumab and the like). In some embodiments, the immunotherapy comprises contacting the cell with at least one or both of a tumor antigen (TA)-targeting antibody (e.g., rituximab, brentuximab, or a combination thereof) and a CD47 blocking antibody (e.g., an anti-CD47 antibody, an anti-SIRPalpha antibody, or any combination thereof).

The antibodies may also be linked to a chemotherapeutic agent. Thus, in some embodiments, the antibody is an antibody-drug conjugate.

### 3. IDENTIFYING REGULATORS OF PHAGOCYTOSIS

Further provided herein are methods for identifying regulators of phagocytosis. The methods may comprise incubating cells with LPS-treated macrophages, wherein the macrophages comprise a CRISPR knockout system or a CRISPR activation (CRISPRa) system and each macrophage comprises at least one guide RNA targeting an endogenous gene; removing unphagocytosed cells from macrophages, and analyzing the unphagocytosed cells or macrophages for indications of which factors regulated phagocytosis.

The methods allow for repetition of any of the disclosed steps. In some embodiments, the incubating and removing steps may be repeated one or more times prior to the analysis of the unphagocytosed cells or macrophages for indications of which factors regulated phagocytosis. Thus, the methods provide for enrichment or changes in selection stringency of unphagocytosed cells or macrophages for indications of which factors regulated phagocytosis by repeating previous steps in the methods.

In some embodiments, the methods identify regulators of antibody-dependent cellular phagocytosis (ADCP) in cells comprising at least one or all; incubating cells with LPS-treated macrophages in the presence of anti-CD20, anti-EGFR, anti-CD30, and/or anti-CD47 antibodies, wherein the cells comprise a CRISPR knockout system or a CRISPR activation (CRISPRa) system and each cell comprises at least one guide RNA targeting an endogenous gene; separating unphagocytosed cells from macrophages; extracting nucleic acids from the unphagocytosed cells; and identifying the guide RNA and guide RNA endogenous gene targets in the unphagocytosed cells. In some embodiments, the cells comprise a CRISPR knockout system and the incubation is in the presence of anti-CD20 antibodies. In some embodiments, the cells comprise a CRISPRa system and the incubation is in the presence of anti-CD20 and anti-CD47 antibodies.

Anti-CD20, anti-EGFR, anti-CD30, and/or anti-CD47 antibodies are well known in the art. For example, anti-CD20 antibodies include, but are not limited to, rituximab and binutuzumab; anti-EGF antibodies include, but are not limited to, cetuximab and necitumumab; anti-CD30 antibodies include, but are not limited to, brentuximab; and anti-CD47 antibodies include, but are not limited to: 5F9, SFR231, and STI-6643.

The methods may further comprise identifying the guide RNA and guide RNA endogenous gene targets in the unphagocytosed cells. In some embodiments, the guide RNA is determined by sequencing or microarray analysis. It should be appreciated that any means of determining nucleic acid sequences is compatible with identifying the guide RNA. Furthermore, the genomic DNA may be extracted and sequenced to identify any genetic modifications resulting from the guide RNA.

The DNA or RNA may be amplified via polymerase chain reaction (PCR) before being sequenced. PCR and sequencing techniques are well known in the art; reagents and equipment are readily available commercially.

Non-limiting examples of sequencing methods include Sanger sequencing or chain termination sequencing, Maxam-Gilbert sequencing, capillary array DNA sequencing, thermal cycle, solid-phase sequencing, sequencing with mass spectrometry such as matrix-assisted laser desorption/ionization time-of-flight mass spectrometry (MALDI-TOF/MS), and sequencing by hybridization, NGS (next-generation sequencing), Polony sequencing, ion semiconductor sequencing, DNA nanoball sequencing, single-molecule real-time sequencing, sequencing by synthesis (e.g., Illumina/Solexa sequencing), sequencing by ligation, sequencing by hybridization, nanopore DNA sequencing, massively Parallel Signature Sequencing (MPSS); pyro sequencing. SOLiD sequencing; shortgun sequencing; Heliscope single molecule sequencing; single molecule real time (SMRT) sequencing; high-throughput sequencing; and/or deep-sequencing.

In some embodiments, the methods identify regulators of phagocytosis in macrophages. The methods may comprise at least one or all of: incubating cells comprising a detectable label with LPS-treated macrophages, wherein the macrophages comprise a CRISPR knockout system or a CRISPR activation (CRISPRa) system and each macrophage comprises at least one guide RNA targeting an endogenous gene; removing unphagocytosed cells from macrophages; and separating the macrophages based on presence or absence of the detectable label (e.g., fluorescence, colorimetric, radioactive). The cells may comprise any number of detectable labels, or the same or different types. Preferably, when more than one detectable label is used, the detection of each label is able to be sensitively and reliably detected without non-specific detection of another label. In some embodiments, the detectable label(s) comprises a fluorescent label.

The gRNA may be a crRNA, crRNA/tracrRNA (or single guide RNA, sgRNA). A gRNA hybridizes to (complementary to, partially or completely) a target nucleic acid sequence (e.g., the genome in a host cell) of an endogenous gene. To facilitate gRNA design, many computational tools have been developed (See Prykhozhij et al. (PloS ONE, 10(3): (2015)); Zhu et al. (PloS ONE, 9(9) (2014)); Xiao et al. (Bioinformatics. Jan. 21 (2014)); Heigwer et al. (Nat Methods, 11(2): 122-123 (2014)). Methods and tools for guide RNA design are discussed by Zhu (Frontiers in Biology, 10 (4) pp 289-296 (2015)), which is incorporated by reference herein. Additionally, there are many publicly available software tools that can be used to facilitate the design of sgRNA(s); including but not limited to, Genscript Interactive CRISPR gRNA Design Tool, WU-CRISPR, and Broad Institute GPP sgRNA Designer. There are also publicly available pre-designed gRNA sequences to target many genes and locations within the genomes of many species (human, mouse, rat, zebrafish, C. elegans), including but not limited to, IDT DNA Predesigned Alt-R CRISPR-Cas9 guide RNAs, Addgene Validated gRNA Target Sequences, and GenScript Genome-wide gRNA databases.

The unphagocytosed cells may be separated from the macrophages using standard methods known in the art including washing with buffers, centrifugation, filtration, density gradients, and the like.

In some embodiments, the cells are cancer cells. The cancer cells may be from any cancer of interest, including carcinoma, sarcoma, lymphoma, leukemia, melanoma, mesothelioma, multiple myeloma, or seminoma. In some embodiments, the cells are lymphoma cells (e.g., Ramos or Karpas-299 lymphoma cells). The cancer cells may be derived from a cancer of the bladder, blood, bone, brain, breast, cervix, colon/rectum, endometrium, head and neck, kidney, liver, lung, lymph nodes, muscle tissue, ovary, pancreas, prostate, skin, spleen, stomach, testicle, thyroid, or uterus. The cancer cells may be primary cells or derived from a cell line.

In some embodiments, the cells lack a regulator of phagocytosis. For example, the cells may naturally lack or have been engineered to lack an endogenous gene determined by methods described herein as a regulator of phagocytosis.

The macrophages may include macrophages derived from any anatomical location or tissue type (e.g., monocyte-derived macrophages or bone marrow-derived macrophages). The macrophages may be classically-activated (M1) macrophages, wound-healing macrophages (also known as alternatively-activated (M2) macrophages), and regulatory macrophages (Mregs). The macrophages may be primary macrophages or a clonal cell line (e.g., THP-1, U937, and J774 macrophage cells).

Bacterial lipopolysaccharide (LPS), the major structural component of the outer wall of Gram-negative bacteria, is a potent activator of macrophages. The macrophages may be treated with any quantity of LPS, for any length of time, prior to the disclosed methods necessary for activation. Activation of the macrophages can be monitored by the production of various cytokines, such as TNFα, IL1β, IL6, IL8, IL10, IL12, IL15, and TGFβ, with methods known in the art.

In some embodiments, the macrophages are treated with LPS at least 12 hours prior to incubation with the cells. For example, the macrophages may be treated with LPS about 12 hours, about 14 hours, about 16 hours, about 18 hours, about 20 hours, about 22 hours, about 24 hours, about 28 hours, about 32 hours, about 36 hours, about 40 hours, about 44 hours, about 48 hours, or more prior to incubation with the cells. In select embodiments, the macrophages are treated with LPS about 24 hours prior to incubation with the cells.

In some embodiments, the macrophages are treated with 1-1000 ng/mL LPS (e.g., about 1 ng/mL, about 5 ng/mL, about 10 ng/mL, about 50 ng/mL, about 100 ng/mL, about 250 ng/mL, about 500 ng/mL, about 750 ng/mL, or about 1000 ng/mL LPS. The macrophages may be treated with 1-10 ng/mL LPS, 1-50 ng/mL LPS, 1-100 ng/mL LPS, 1-200 ng/mL LPS, 1-300 ng/mL LPS, 1-400 ng/mL LPS, 1-500 ng/mL LPS, 1-600 ng/mL LPS, 1-700 ng/mL LPS, 1-800 ng/mL LPS, 1-900 ng/mL LPS, 10-50 ng/mL LPS, 10-100 ng/mL LPS, 10-200 ng/mL LPS, 10-500 ng/mL LPS, 10-700 ng/mL LPS, 10-1000 ng/mL LPS, 50-100 ng/mL LPS, 50-200 ng/mL LPS, 50-500 ng/mL LPS, 10-700 ng/mL LPS, 10-1000 ng/mL LPS, 100-200 ng/mL LPS, 100-500 ng/mL LPS, 100-700 ng/mL LPS, 100-1000 ng/mL LPS, 200-500 ng/mL LPS, 200-700 ng/mL LPS, 200-1000 ng/mL LPS, 500-700 ng/mL LPS, 500-1000 ng/mL LPS, or 700-1000 ng/mL LPS.

### 4. EXAMPLES

### Materials and Methods

Cell Culture Ramos, Karpas-299, NCI-H82 and K562 cells were maintained in suspension culture in T-150 flasks for library propagation and tissue culture plates for single-gene knockout lines, all in RPMI-1640 supplemented with 2 mM glutamine, 100 units ml⁻¹ penicillin, 100 mg ml⁻¹ streptomycin and 10% FCS. J774 cells were cultured in 15 cm plates for screens and library preparation in DMEM supplemented with 2 mM glutamine, 100 units ml⁻¹ penicillin, 100 mg ml⁻¹ streptomycin and 10% FCS and were passaged when nearing confluency by scraping. HeLa, HCT-116, RKO, NCI-H23, SKBR3, and OVCAR8 cells were passaged with Accutase. All cells were cultured in a humidified 37° C. incubator set at 5% CO₂ and passaged two to three times weekly. To generate frozen aliquots, cells were pelleted by centrifugation (300 g, 5 min, room temperature), suspended in 90% FCS and 10% dimethylsulfoxide (DMSO), and frozen in cell freezing containers at −80° C. overnight before transfer to liquid nitrogen for long-term storage.

Genome-wide CRISPR screens in Ramos cells For the CRISPR knockout screen in Ramos cells, a genome-wide, 10 sgRNA per gene CRISPR deletion library was synthesized, cloned, and infected into Cas9-expressing Ramos cells. Briefly, ˜300 million Ramos cells stably expressing SFFV-Cas9-BFP were infected with the CRISPR knockout library at a multiplicity of infection (MOI) of ˜0.2. Cells expressing sgRNAs were selected for using puromycin (1 μg ml⁻¹) for 3 d such that >90% of cells were mCherry-positive as measured by flow cytometry. Selected cells were then allowed to recover and expand in puromycin-free media for up to 7 d. For the CRISPRa screen, a clonal CRISPRa-VPR-expressing line was first constructed by transducing wild-type Ramos cells with a lentiviral construct expressing CRISPRa-VPR and GFP. Cells expressing low levels of GFP were sorted into single cell clones and active clones were identified based on induction of CD2 expression following transduction with an sgRNA targeting the CD2 TSS. One active clone was selected for subsequent experiments based on the degree of CD2 induction as well as its similar growth rate and phagocytosis susceptibility compared to the parental cell population. As with the CRISPR knockout screen, a genome-wide, 10 sgRNA per gene library was synthesized, cloned, and infected into CRISPRa-expressing Ramos cells and selected with puromycin as above.

For the screens, cells were split into two conditions, each in duplicate: an untreated control group and ADCP treated group. In the ADCP group, for each round of treatment, Ramos cells were incubated with J774 macrophages, which had been seeded into 15 cm plates 48 h prior (at 5 M cells per plate) and treated with LPS (10 ng ml⁻¹) starting 24 h prior, in the presence of anti-CD20 antibodies (50 ng ml⁻¹) (CRISPRko screen) or both anti-CD20 antibodies (50 ng ml⁻¹) and anti-CD47 antibodies (100 ng ml-1) (CRISPRa screen). 25 M Ramos cells were added to each plate and incubated with J774 macrophages for 24 h, and were then removed from the macrophage-containing plates via two rounds of washing with complete RPMI media. Ramos cells were then allowed to recover in T-150 flasks for 24-72 h before the next round of treatment. At the end of the screen, 300 million cells were recovered from each condition and pelleted by centrifugation. Genomic DNA of each condition was extracted using Qiagen DNA Blood Maxi kit (catalog no. 51194). The sgRNA sequences were amplified and sequenced using an Illumina NextSeq with ˜40 million reads per condition; ˜200× coverage per library element). Analysis and comparison of guide composition of ADCP treated versus untreated conditions were performed using casTLE as previously described.

Genome-wide CRISPR knockout screens in Karpas-299 cells were performed similarly to the genome-wide screens in Ramos cells.

Genome-wide macrophage screens For the CRISPR knockout screen in J774 cells, a genome-wide, 10 sgRNA per gene CRISPR mouse deletion library was synthesized and cloned and infected into Cas9-expressing J774 cells. Following puromycin selection, the genome-wide was cultured for 7-10 d, then plated in 15 cm tissue culture dishes at a starting density 5M cells per plate. After 24 h, media was replaced with DMEM containing 10 ng ml⁻¹ LPS. After a further 24 h, macrophages were incubated with a mixture of calcein-labeled Safe^{KO} Ramos cells and CellTrace Far-red-labeled APMAP^{KO} macrophages for 24 h. Unphagocytosed Ramos cells were removed by extensive washing with PBS. Macrophages were lifted by scraping, washed twice in PBS, concentrated, and separated into four populations on an Aria flow cytometer. 80M cells were collected and used for genomic DNA extractions, PCRs, and sequencing, as above.

Generation of cell lines For generating individual sgRNA-expressing cell lines, Ramos, Karpas-299, HeLa, HCT-116, NCI-H23, RKO, K562, SKBR3, NCI-H82 cells that express EF1Alpha-Cas9-BFP were infected with lentiviral constructs expressing a given sgRNA along with puromycin or blasticidin resistance cassette. At 3 d after infection selection was done with 1-2 μg ml⁻¹ puromycin or 10 μg ml⁻¹ blasticidin for 3 d.

Plasmids To generate APMAP expression constructs to reconstitute in APMAP knockout cell lines, the human APMAP open reading frame, with a 3×FLAG tag appended to the C-terminus, was synthesized (Twist Biosciences) and cloned into a TOPO backbone vector. Point mutations were installed using site-directed mutagenesis, and the APMAP-FLAG region was then subcloned into pMCB394, a lentiviral expression vector.

For the Δcyto truncation, the region encoding APMAP residues 62-416 was amplified with a new start codon and cloned into pMCB394. The chimeric constructs were created by amplifying the regions encoding the cytoplasmic and transmembrane domains of two single-pass transmembrane proteins with similar topology to APMAP: POMK (residues 1-43) and TFRC (residues 1-88, with the first 6 residues encoded in the primer to change them from the endogenous sequence to MSRRRS, mutations that were shown to retain TFRC primarily in the endoplasmic reticulum) and fusing them via overlap extension PCR to the region encoding APMAP residues 62-416. The TFRC^{RR} allele was used in place of TFRC^{WT}, as the TFRC^{WT}-APMAP-FLAG chimera was not detectable by western blot.

Time-lapse microscopy assay for phagocytosis J774 cells were lifted by scraping, counted, and plated in 24 well tissue culture plates at a density of 100,000 cells per well in 0.5 ml media 48 h prior to the start of the experiment. At 24 h after plating, media was aspirated and replaced with fresh media containing 10 ng ml⁻¹ lipopolysaccharide (LPS) (Sigma). On the day of the experiment, target cells that had been transduced with sgRNAs, co-expressed with GFP and PuroR, and selected with puromycin as described above, were lifted, pelleted, and counted. Cells were washed three times in dPBS, incubated in dPBS containing 6.6 ng ml-1 pHrodo-Red succinimidyl ester (Thermo Fisher) at a cell concentration of 1M ml⁻¹ for 30 minutes, washed once, and resuspended in DMEM supplemented with 2 mM glutamine, 100 units ml⁻¹ penicillin, 100 mg ml⁻¹ streptomycin and 10% heat-inactivated FCS, and containing anti-CD20, anti-CD30, and/or anti-CD47 antibodies, as indicated, at a cell concentration of 1M ml⁻¹. 220 μl of cell suspensions was added to each well. Plates were transferred to an incubator and imaged every 30 or 60 minutes using an Incucyte (Essen). Total red intensity for each well, averaged over 16 images per well, was calculated after applying a threshold for red intensity that excluded most or all cells at the first time point using top-hat background subtraction. Reported values represent the mean total red fluorescence intensity at each timepoint, minus the total red fluorescence at the initial timepoint (to remove signal from occasional autofluorescent debris), normalized to the initial total green fluorescence signal (to account for small variations in target cell density), of duplicate, triplicate, or quadruplicate wells, as indicated in the legend.

Mouse tumor xenografts For mouse tumor experiments, tumor xenografts were established by injection of Safe^{KO} and APMAP^{KO} Ramos or NCI-H82 cells into the flank of male NSG mice. In brief, 4×10⁶ cells were injected per mouse. Mice were injected intraperitoneally with anti-Cd47 daily (400 μg), and tumor size was measured using calipers. Tumor size was measured once every 2 days in each mouse for an additional 12-17 days. The number of mice represented in the final analyses was at least 5 in each group.

No statistical tests were used to calculate sample size. Sample size was at least 7 for each treatment group to account for differences in tumor formation and growth, and to ensure recovery of a sufficient quantity of mice with tumors of approved size at each time point of the study. Following injection of tumor cells, the mice were randomly assigned into two treatment groups for PBS or anti-CD47 injection. Fold change in tumor volume was statistically analyzed using the unpaired, two-way t-test. Blinding was not possible because the experiments were performed by a single researcher. Animals were euthanized when the xenograft tumor size reached two centimetres in any two dimensions. No mouse exhibited severe loss of body weight (>15%) or evidence of infections or wounds.

Immunoblotting Cleared cell extracts prepared in lysis buffer (50 mM Tris-HCl pH 7.5, 150 mM NaCl, 1 mM EDTA, 1% Triton X-100, 1×cOmplete protease inhibitor cocktail (Roche) were heated in SDS loading buffer and subjected to SDS-PAGE, transferred to nitrocellulose, blotted, and imaged using an Odyssey CLx (LI-COR Biosciences) or Supersignal West Femto Maximum Sensitivity Substrate with a Chemidoc System (Bio-Rad). Cell pellets were resuspended directly in SDS loading buffer, sonicated, and loaded on SDS-PAGE. Extracts were first treated with PNGase F (NEB) following the manufacturer's instructions. The following antibodies were used: mouse monoclonal anti-APMAP (OTI4F6, Origene, 1:2000 dilution), mouse monoclonal anti-FLAG (clone M2, F1804, Sigma, 1:2000 dilution), mouse monoclonal anti-GAPDH (clone 6C5, AM4300, Fisher, 1:5000 dilution), and rabbit polyclonal anti-beta actin (ab8227, Abcam, 1:2000 dilution).

Flow cytometry Ramos cells were analyzed by flow cytometry using the following antibodies: anti-CD20-APC (clone 2H7, Biolegend), anti-CD2-APC (clone REA972, Mitenyi Biotech), anti-CD47 (clone B6.H12, BioXCell), and anti-Calreticulin-DyLight-488 (FMC 75, Enzo Life Sciences), and with Annexin-V-FITC (A13199, Thermo Fisher), or with biotinylated Maackia amurensis lectin II (MAL-II) (B-1265, VectorLabs) followed by streptavidin-APC (Thermo Fisher). Sialidase (neuraminidase from Vibrio cholerae, Sigma Cat. No. 11080725001) treatment was performed by incubating cells with 30 nM enzyme in dPBS at a cell concentration of 1 million ml-1 for 1 h at 37 deg. Single cell suspensions were prepared from diced tumors and fixed in paraformaldehyde as described previously. Cells were stained with Zombie-NIR viability dye (BioLegend), anti-CD45 (clone 30-F11, BioLegend), anti-F4/80 (clone BM8, BioLegend), and anti-CD11b (clone M1/70, BioLegend) and analyzed using an Acea Novocyte Quanteon flow cytometer and FlowJo software (version 10.6.1). Live, CD45⁺ cells were counted using gates that separated the CD45⁻ and CD45⁺ populations, and macrophages were counted using a single gate applied equally to all samples.

Isolation of human peripheral blood mononuclear cell derived macrophages Primary human donor-derived macrophages were isolated and cultured following a previously described protocol (et al., Proc. Natl. Acad. Sci. U.S.A. 118, (2021), incorporated herein by reference in its entirety) with minor modifications. Leukocyte reduction chambers were obtained from de-identified healthy donors from the Stanford Blood Center. The protocol was approved by the Stanford University Administrative Panel on Human Subjects in Medical Research and all donors provided informed consent. Cells were separated using Ficoll-Paque gradient centrifugation. Monocytes were then isolated via their adhesion to tissue culture plastic in serum-free RPMI-1640, and subsequently differentiated into macrophages in RPMI-1640 containing 20% heat-inactivated FCS containing 20 ng ml-1 human M-CSF (Peprotech) for 5 d, then lifted with Accutase and manual scraping, and allowed to adhere overnight in 24 well plates. Macrophages were then treated for 48 h with 100 ng ml-1 LPS before use in phagocytosis assays.

Flow cytometry assays for phagocytosis Ramos target cells were stained with calcein, AM (500 ng ml-1, Thermo Fisher) or CellTrace Far Red dyes (1:2000 dilution of stock prepared according to manufacturer's instructions) for 10 min in PBS at 1×10⁶ cells ml⁻¹, washed twice with DMEM containing 10% FCS, and incubated for 24 h with LPS-treated J774 macrophages prior to analysis by flow cytometry (BD Accuri C6). Ramos CD47^{KO} target cells were stained with calcein, AM (500 ng ml-1, Thermo Fisher) and APMAP^{KO} cells were stained with CellTrace Far Red dyes (1:2000 dilution of stock prepared according to manufacturer's instructions) for 10 min in PBS at 1×10⁶ cells ml⁻¹, washed twice with DMEM containing 10% FBS, mixed together with anti-CD20 (500 ng/μl), and incubated for 24 h with LPS-treated J774 macrophages prior to analysis by flow cytometry (BD Accuri C6).

Target cell-macrophage adhesion assay To measure antibody-dependent binding between J774 macrophages and Ramos target cells, macrophages were plated in 24 well tissue culture plates at a density of 100,000 cells per well in 0.5 ml media 48 h prior to the start of the experiment and stimulated with LPS 24 h before the experiment as above. On the day of the experiment, media was aspirated and replaced with DMEM containing 1 μg ml-1 cytochalasin D (Sigma Cat. #C8273), and incubated for 10 minutes at room temperature. Ramos cells (200-500,000) were then added to wells and incubated for 2 h at 37° C. Plates were imaged before and after three washes with PBS to determine the abundance of GFP⁺ Ramos cells (as measured by integrated green intensity) using an Incucyte (Essen) using a 10× objective.

Cell survival analyses To measure cell survival during co-culture with macrophages, two methods were used. mCherry+ Ramos cells expressing indicated sgRNAs were mixed with an equal number of GFP+ Ramos control cells, and incubated with LPS-treated J774 macrophages in the presence of anti-CD20. The percentage of mCherry+ Ramos cells, of all live Ramos cells, was quantified for each cell line, and normalized to the percentage of mCherry+ Ramos control cells, which expressed a non-targeting sgRNA. Survival was also measured by quantifying the total GFP signal per well before and after incubation of GFP+ target cells with LPS-treated J774 macrophages (GFP fluorescence is quenched following uptake of target cells and delivery to the lysosome).

Transcriptome analysis J774 cells were seeded in triplicate at a density 1×10\hat{ }6 cells per 10 cm plate. 24 h later, cells were either harvested (untreated condition) or media was replaced with media containing 100 ng ml⁻¹ LPS for 24 h. At harvest, cells were lysed in RLT buffer and RNA was isolated using the RNeasy Micro Kit (Qiagen). cDNA libraries were prepared using a TruSeq Stranded mRNA Kit (Illumina) and sequenced using an Illumina NextSeq with ˜25 million reads per condition. Transcripts were mapped using STAR (v2.7.0) and gene-level counts were generated using HTSeq (v.0.13.5), followed by differential gene expression analysis using DESeq2 (1.28.1).

Viability Measurements, Ramos cells were plated in 96-well plates in triplicate at a starting density of 50,000 cells per well. Drugs were then added to wells at indicated final concentrations. Cell viability was determined by measuring the phase confluence at 72 h using an Incucyte (Essen). B16-F10 cells were seeded in 24 well plates and phase confluence was measured every 8 h for 6 d.

Confocal microscopy HeLa cells were transduced with a lentiviral construct co-expressing APMAP-FLAG and BlastR, selected with blasticidin and cultured in glass-bottom 24-well plates. Cells were fixed with paraformaldehyde and probed with anti-FLAG (clone M2, Sigma, Cat. #F1804, 1:100 dilution) and anti-calnexin (Abcam Technologies, Cat. #ab22595, 1:1000 dilution)) primary antibodies and imaged using an inverted Nikon Eclipse Ti-E spinning disk confocal microscope using NISElements software (v4.4, Nikon) and an Andor Ixon3 EMCCD camera using an oil-immersion 100× objective (NA=1.45). Images were assembled and adjusted for brightness and contrast in Photoshop CS6 (Adobe).

Analysis of differential expression in TCGA Differential gene expression in 23 TCGA tumor studies was calculated for each gene by comparing gene expression in each tumor study to normal tissue and calculating the fold change.

Analysis of single-cell RNA sequencing studies Analysis of GPR84 expression in human tumor samples was performed using four single-cell RNA sequencing datasets. scRNAseq pre-processing, cell type assignment, t-SNE embedding, and gene expression quantification were performed as described previously.

Homology model of APMAP Using Swiss-Model (template library SMTL version 2020-07-22, PDB release 2020-07-17) with Uniprot accession code for APMAP (Q9DHC9) as input, 50 template structures were retrieved up to a GMQE* score of 0.25. A parallel search was done using FUGUE (version 2.0). Swiss-Model was used with Strictosidine synthase from Rauvolfia serpentina (STR1; sequence similarity=34%; PDB accession ID: 6nv5, unpublished) and serum paraoxonase-1 (PON1; sequence similarity=29%; PDB accession ID: 3sre) first as the highest scoring template structures. Modelling was done on the soluble region, residues 61-407, of APMAP, using the PON1 structure. The automatically generated model was completed by assigning it the 30 N-terminal residues from Drp35 from Staphylococcus aureus (PDB accession ID: 2dg1). The modelling resulted in a 6-blade beta-propeller structure, which was manually inspected in COOT (Version 0.7 (revision 4459)), and the coordinates refined against reference parameters with REFMAC5 (Version 5.8.0135) until deviation from ideal bond length, bond angle, planar restraints, chiral volume, reached convergence and Ramachandran outliers were minimized (1.7% (5 of 347) of the total residues were outliers).

Syngeneic mouse model For syngeneic mouse tumor experiments, tumors were established by injection of either Safe^{KO} or APMAP^{KO} B16-F10 cells into the flank of 8-12 week old female C57BL/6J mice (Jackson Laboratories). In brief, 5×10\hat{ }5 cells were injected in a 100 ul suspension, consisting of 25% Matrigel Basement Membrane Matrix (Corning) and 75% RPMI (Life Technologies). Tumors were established for 5 d, and on Day 5, engraftment outliers were removed as determined by Graphpad Prism Outlier Calculator and mice were randomized into treatment and isotype control groups. Starting on Day 5 post-engraftment, mice were injected intraperitoneally with either anti-TRP1 or isotype control (mouse IgG2a) antibodies (BioXCell, 250 μg) every other day, following a dosing regimen used previously (Sockolosky, J. T. et al. Proc. Natl. Acad. Sci. U.S.A. 113, incorporated herein by reference in its entirety). Tumor size was measured in two dimensions using precision calipers twice weekly for the duration of the experiment. Tumor volumes were calculated by approximating tumors as ellipsoids with two radii, x and y, where x is the largest measurable dimension of the tumor and y is the dimension immediately perpendicular to x, using the formula: volume=4/3π×(x/2)²×(y/2). The number of mice represented in the final analysis was 6-7 for all groups. No statistical tests were used to calculate sample size. Starting sample size was n=7-8 for each treatment group to account for differences in tumor engraftment and growth, and to ensure recovery of a sufficient quantity of mice with tumors of approved size at each time point of the study. Data are presented up to day 20 (when sufficient mice were still alive to analyze the full randomized cohort), and full data (until day 25) are included as Source Data. Change in tumor volume was evaluated using two-way ANOVA in GraphPad Prism. Blinding was not possible because the experiments were primarily performed by a single researcher. All mouse experiments were conducted in accordance with the guidelines of the Institutional Animal Care and Use Committees (IACUC) of Stanford University. Animals were euthanized once several of the largest xenograft tumors had reached 2000 mm³. No mouse exhibited severe loss of body weight (>15%) or evidence of infections or wounds. Full data are included as Source Data.

### Example 1

### Genome-Wide CRISPR Screens Identify Regulators of ADCP

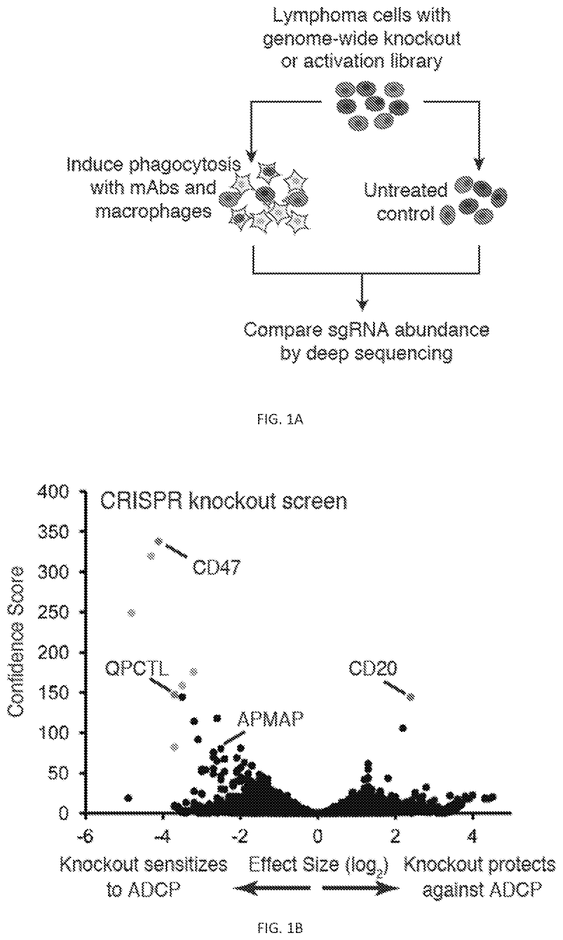

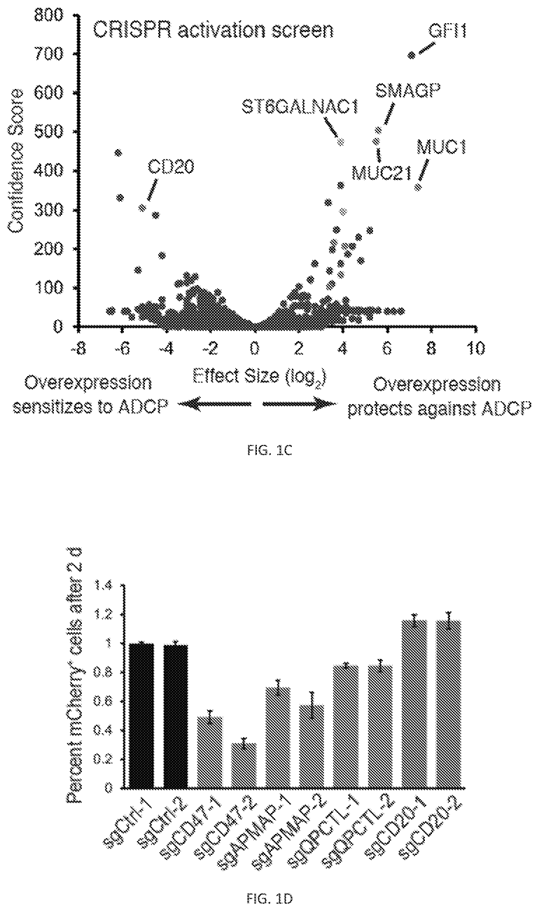

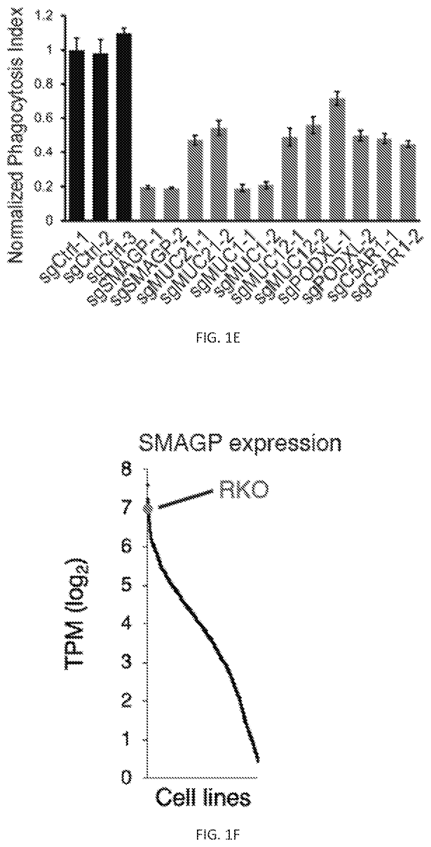

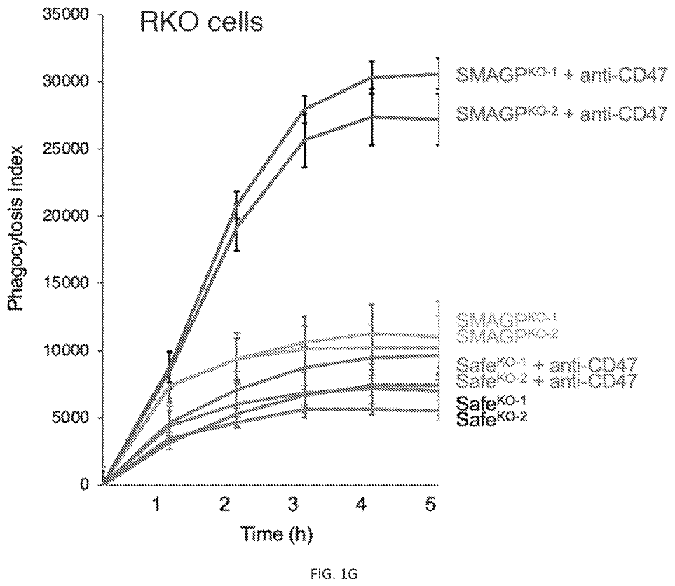

To establish a scalable platform for genetic screening for regulators of ADCP, conditions for antibody-stimulated uptake of cancer cells by macrophage cell lines were optimized, which, unlike primary macrophages, can readily be cultured at the ultra-high scales necessary for screening. In pilot assays, the J774 mouse macrophage cell line exhibited dramatically higher rates of ADCP than other available macrophage cell lines, including human U937 cells, and was thus selected for screening. To conduct genome-wide screens for cancer cell factors that regulate susceptibility to ADCP, a genome-wide knockout pool of Ramos Burkitt lymphoma cells was first generated by stably expressing Cas9 and introducing a CRISPR knockout library containing 10 sgRNAs targeting every protein-coding gene. The Ramos genome-wide knockout pool was then incubated with rituximab-biosimilar anti-CD20 antibodies in the presence of LPS-activated J774 macrophages, a treatment found to drive high rates of phagocytosis. By sequencing the population of sgRNAs in the knockout cell population after two rounds of macrophage-mediated killing and comparing the relative abundance of sgRNAs in the treated and untreated cell populations, genes whose absence specifically affects the susceptibility of Ramos cells to ADCP were identified (FIG. 1A).

The strongest sensitizing hit in this screen was CD47, reflecting its well-characterized role in suppressing phagocytosis (FIGS. 1B, 5A, and 14A). The glutaminyl cyclase QPCTL was also recovered as a strong sensitizing hit (FIG. 1B), consistent with its recently described function as an essential post-translational regulator of CD47 enabling CD47 interaction with SIRPA. Several genes required for the synthesis of sialic acids were also uncovered as strong sensitizing hits (FIG. 1B) concordant with the well-documented role of sialic acid-modified cell surface proteins and lipids in regulating macrophage function. Additionally, the target of the antibody used to drive ADCP, CD20/MS4A1 was the top protective hit (FIG. 1B), validating the ability to detect positive and negative regulators of ADCP susceptibility. Preliminary validation experiments of these genes and several of the top hits was performed using competitive growth assays in the presence and absence of macrophages, and it confirmed ADCP phenotypes measured in the bulk screen in individual assays (FIG. 1D). Taken together, these results validate the ability of this screening platform to identify bonafide regulators of cancer cell susceptibility to ADCP.

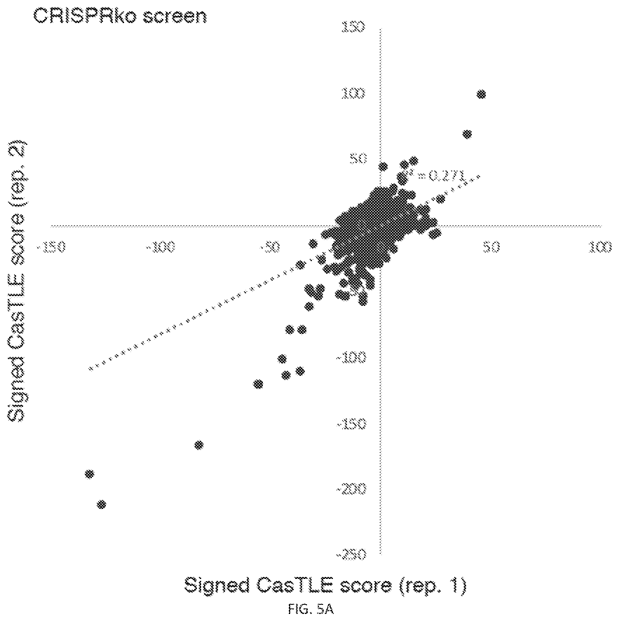

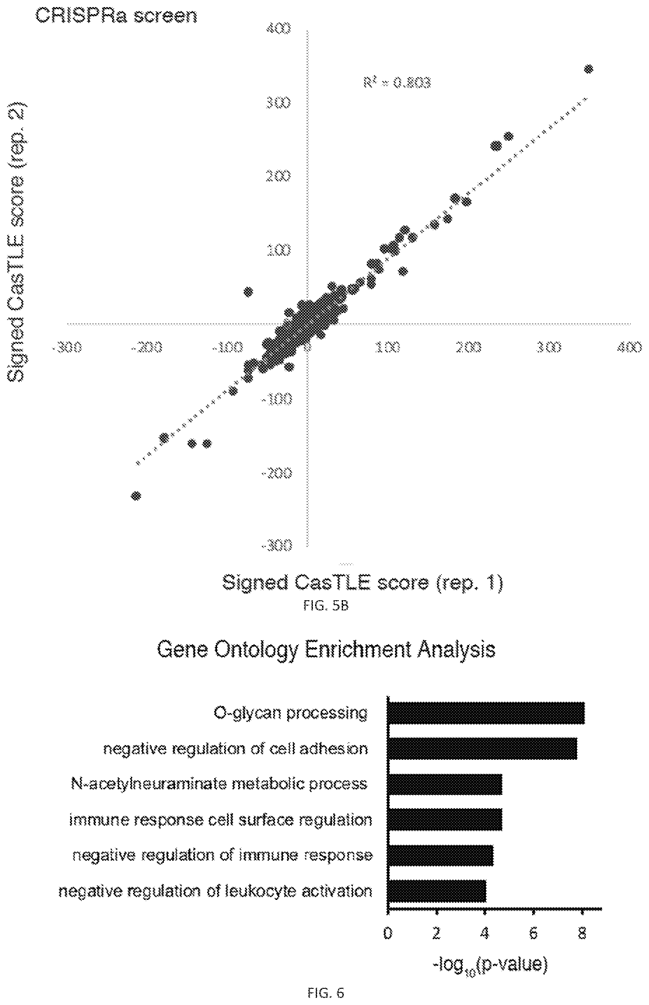

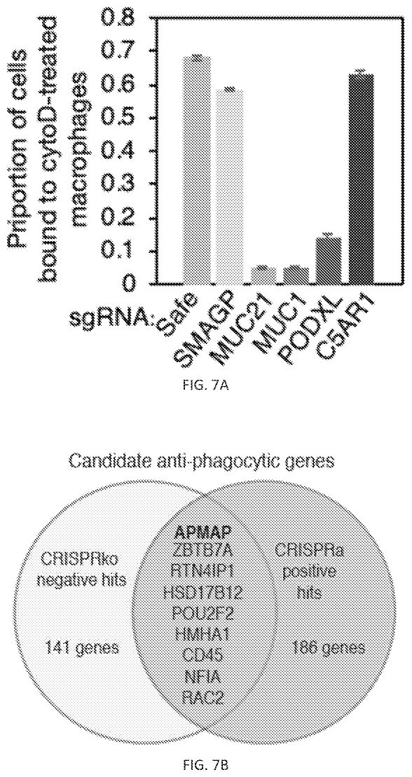

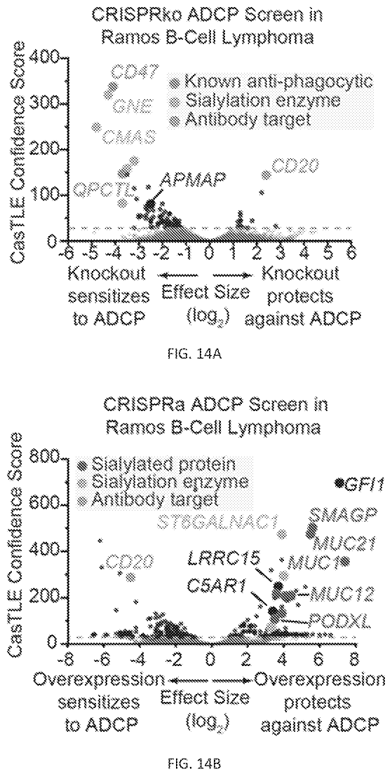

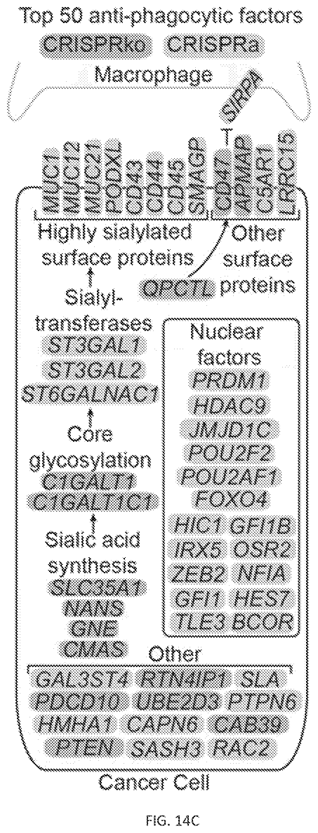

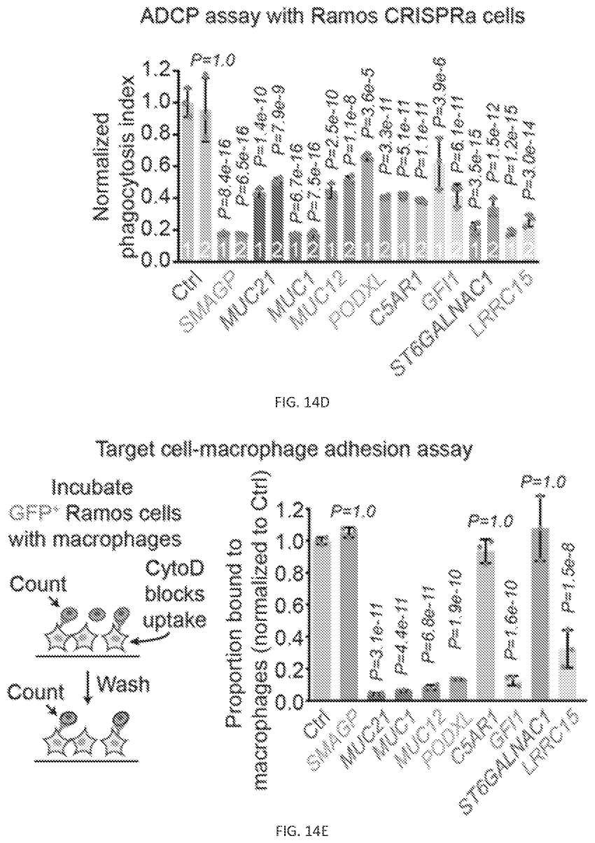

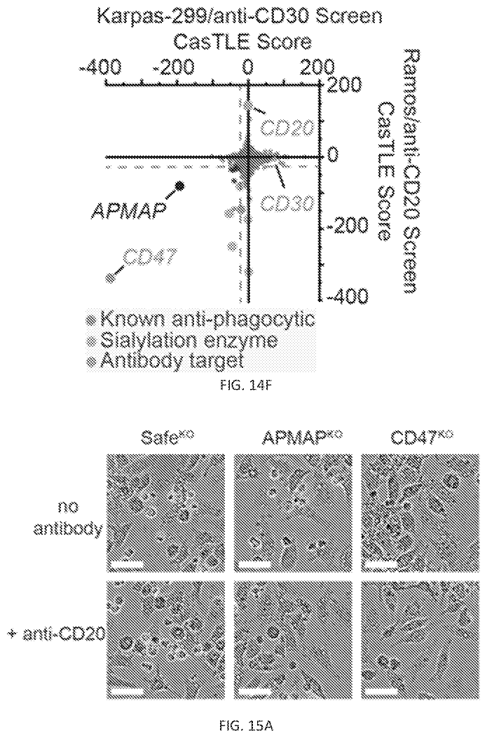

Because the expression of many anti-phagocytic genes is restricted to only a subset of cancer types, it was unknown whether CRISPRa screens could be used to uncover additional regulators of ADCP that are not endogenously expressed at high levels in Ramos cells, but which can be “displayed” on the cancer cell surface upon overexpression. To this end, Ramos cells were stably transduced with a CRISPRa construct and a genome-wide activation library again containing 10 sgRNAs targeting each protein-coding gene. The Ramos activation library was subjected to multiple rounds of selection by ADCP using a similar approach as before (FIGS. 1A and 5B), but included anti-CD47 antibodies to block the function of CD47, which was found to be the most potent anti-phagocytic factor expressed by this cell line, to better enable detection of other anti-phagocytic factors. Overexpression of CD20 strongly sensitized Ramos cells to ADCP (FIGS. 1C and 14B). Many of the top protective hits (e.g., genes that increase survival of cells upon incubation with macrophages and anti-CD20 and anti-CD47 antibodies) were either involved in sialylation or were mucins or other cell surface proteins that are known to be heavily modified with sialic acids (FIG. 6), several of which have previously been proposed to regulate phagocytosis. Interestingly, several of these genes have been described as cancer antigens (e.g., MUC1, LRRC15) or as markers of metastasis (e.g., PODXL23, SMAGP), but have unclear roles in cancer development. Several of these genes were validated as regulators of susceptibility to ADCP in individual experiments (FIGS. 1E and 14D). Using a cell adhesion assay, overexpression of SMAGP and C5AR1 did not affect binding of cells to macrophages whereas overexpression of several mucin and mucin-like genes prevented macrophages from binding Ramos target cells in the presence of rituximab, suggesting that the screen paradigm is capable of identifying multiple classes of ADCP regulators (FIG. 7A). Furthermore, to test the relevance of these hits to the ADCP sensitivity of cells in which these genes are endogenously expressed at high levels, the Cancer Cell Line Encyclopedia gene expression database was queried to identify cell lines with the highest expression of these genes. SMAGP, a poorly characterized cell surface glycoprotein which was among the top hits in the CRISPRa screen was found to be expressed at high levels in several colon cancer cell lines, including RKO cells (FIG. 1F). To test whether SMAGP protects RKO cells against phagocytosis, RKO cells were transduced with Cas9 and sgRNAs targeting SMAGP, and deletion of SMAGP strongly sensitized RKO cells to phagocytosis (FIG. 1G).

These screens thus uncovered numerous known and novel regulators of cancer cell susceptibility to phagocytosis. APMAP (Adipocyte Plasma Membrane Associated Protein) was among the strongest modifiers of sensitivity to phagocytosis identified in the Ramos CRISPRko screen. APMAP was also one of ten genes that was uncovered as both a sensitizing hit in the CRISPRko screen and as a protective hit in the CRISPRa screen (FIG. 7B), suggestive in both cases of a role in mediating cancer cell resistance to ADCP. APMAP is known to be expressed ubiquitously in nearly all tissue types and has also been found to be overexpressed in a variety of malignancies, indicative of its possible functional relevance to a range of cancer types. APMAP is also known to contain an enzymatic domain related to human serum paraoxonases, indicated that it provides a druggable therapeutic target.

### Example 2

### APMAP Loss Synergizes with Tumor-Antigen Targeting mAbs to Sensitize Lymphoma Cells to ADCP

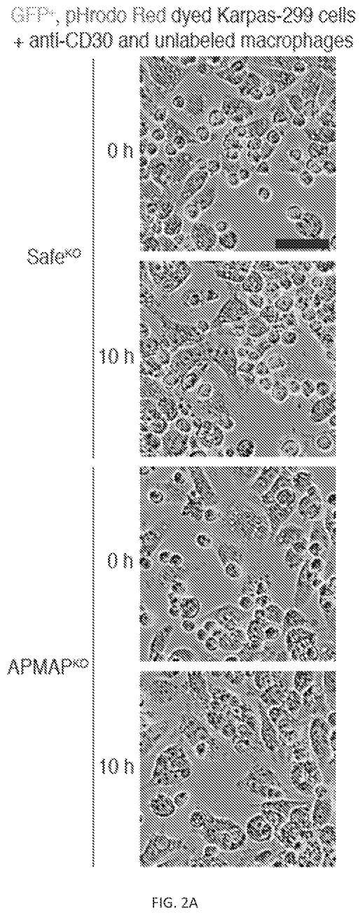

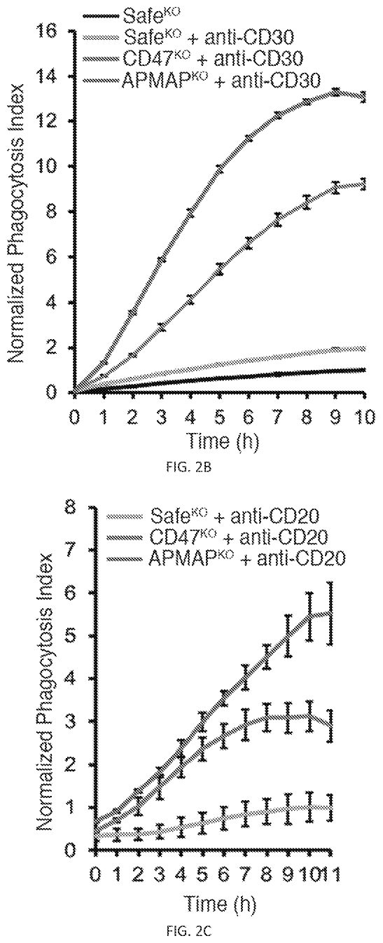

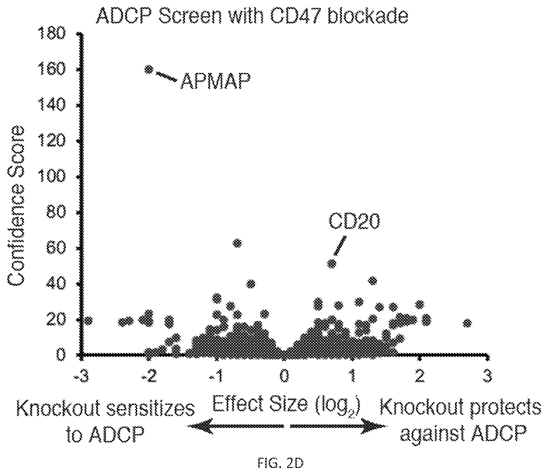

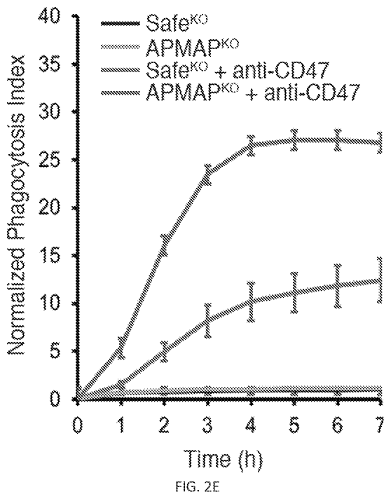

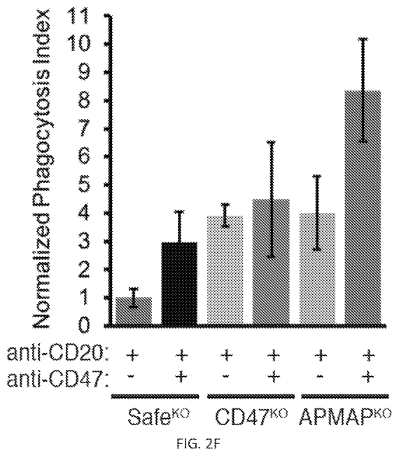

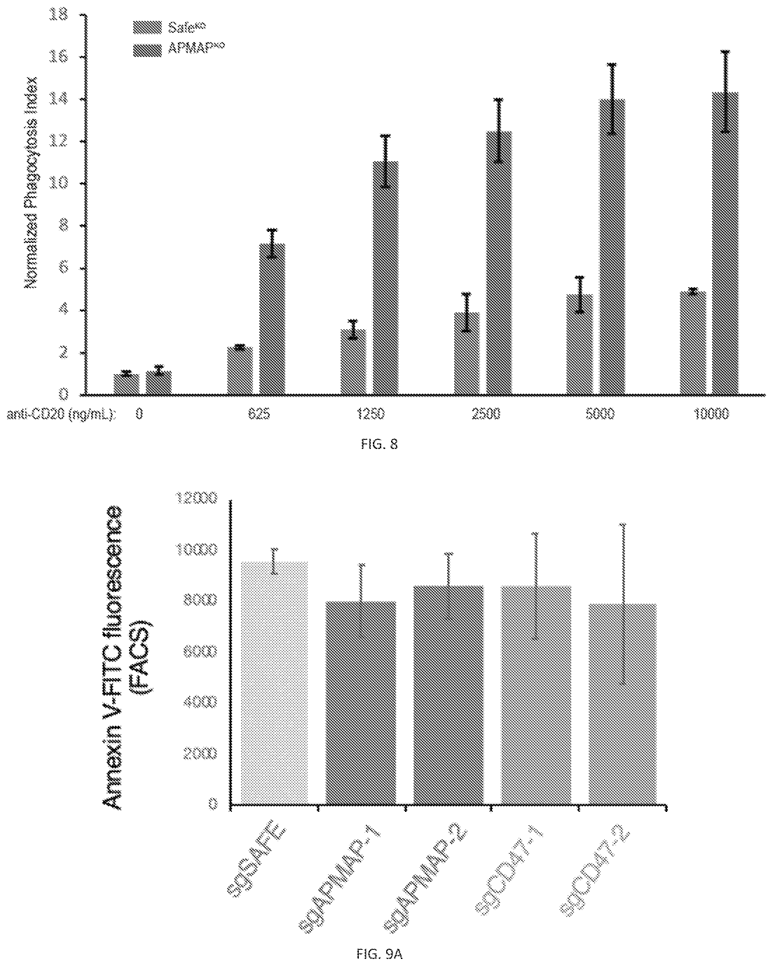

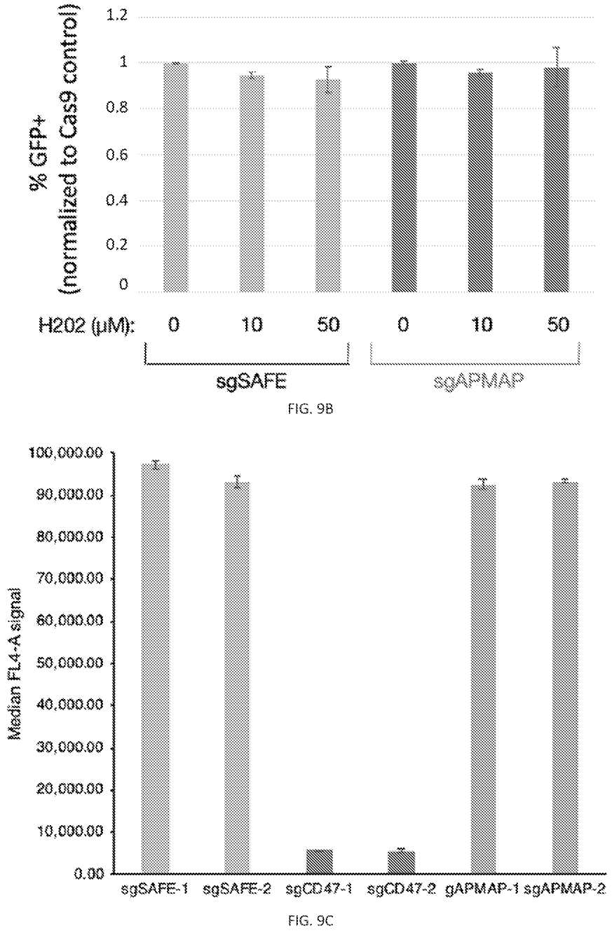

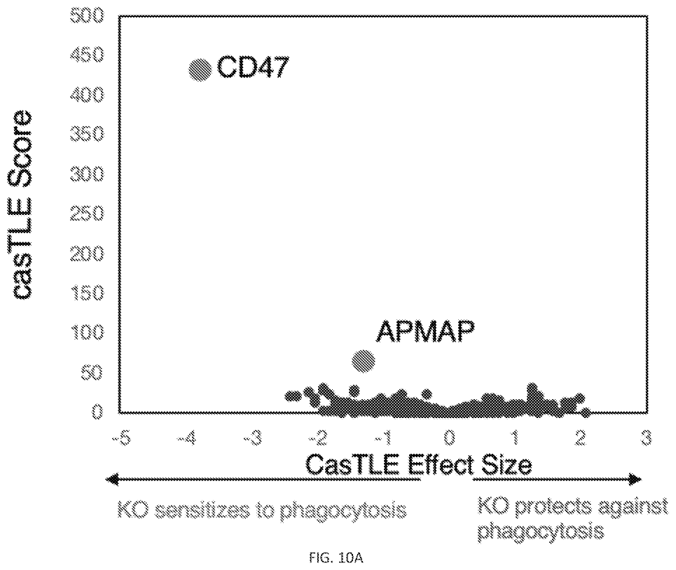

Quantification of cancer cell phagocytosis using time-lapse microscopy revealed a significant increase in the sensitivity of APMAP knockout (APMAP^{KO}) Ramos cells to phagocytosis by J774 macrophages in the presence of anti-CD20 antibodies, consistent with the phenotypes measured in the context of the genome-wide screen (FIG. 8). To test whether APMAP protects other lymphoma cells against phagocytosis, the susceptibility of Karpas-299 anaplastic large cell lymphoma cells to phagocytosis was examined. APMAP^{KO} Karpas-299 cells exhibited significantly enhanced susceptibility to phagocytosis in the presence of anti-CD30 antibodies (FIGS. 2A-2B). To confirm that the effect of APMAP on cancer cell sensitivity to ADCP was not specific to mouse macrophages, the sensitivity of Ramos cells to phagocytosis driven by anti-CD20 mAbs was monitored in the presence of human U937 macrophages. APMAP^{KO} cells were strongly sensitized to phagocytosis (FIG. 2C). APMAP^{KO} Ramos cells did not exhibit measurable deficiencies in growth rate, steady-state cell viability, or the surface expression of the known “eat-me” signal phosphatidylserine compared to Safe^{KO} cells (FIG. 9A). Additionally, Ramos APMAP^{KO} cells did not exhibit detectable differences in sensitivity to oxidative stress or cell surface expression of CD47 when compared to Safe^{KO} cells (FIGS. 9B-9C). Thus, APMAP was a potent suppressor of ADCP in lymphoma cells; notably, the magnitude of the effect of APMAP loss on susceptibility to ADCP was similar to or greater that of CD47 loss (FIGS. 2B-2C).

### Example 3

### APMAP Loss Synergizes with CD47 Blockade to Enhance Cancer Cell Phagocytosis

The poorly characterized gene APMAP was among the strongest modifiers of sensitivity to phagocytosis identified in both the Ramos CRISPRko screen (FIG. 14A) and a second genome-wide ADCP screen we conducted in Karpas-299 T lymphoma cells using anti-CD30 antibodies (FIG. 14F). APMAP is ubiquitously expressed in human tissue types and in all cell lines of the Cancer Cell Line Encyclopedia, and is overexpressed in several malignancies. Nonetheless, APMAP is not essential for growth under standard culture conditions in any of the 789 lines profiled in DepMap or for mouse viability, implying that targeting APMAP would not exhibit broad toxicity.

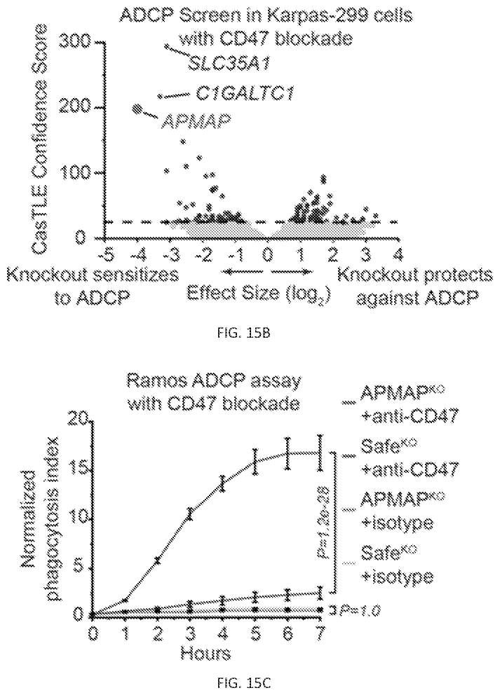

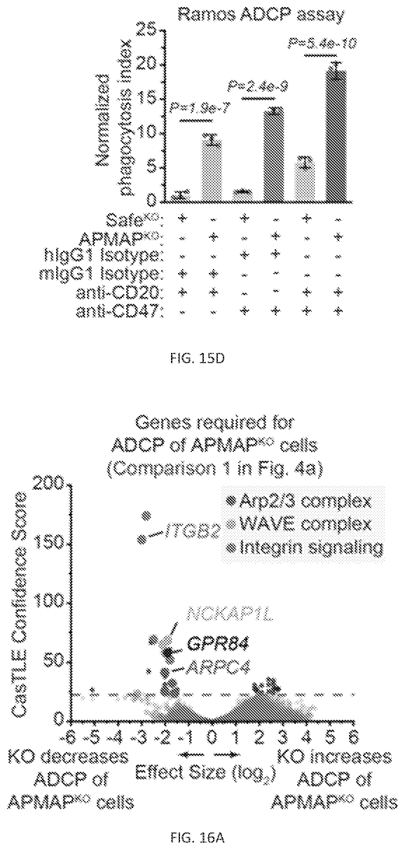

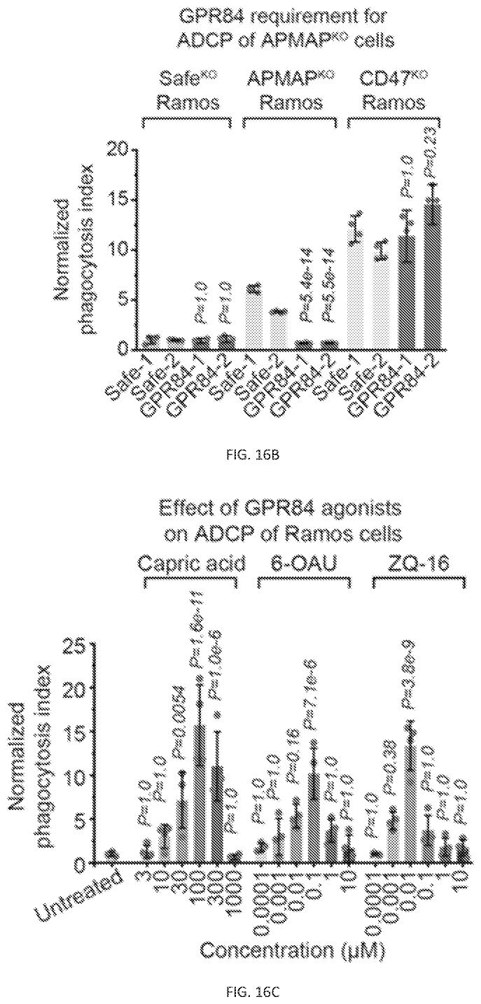

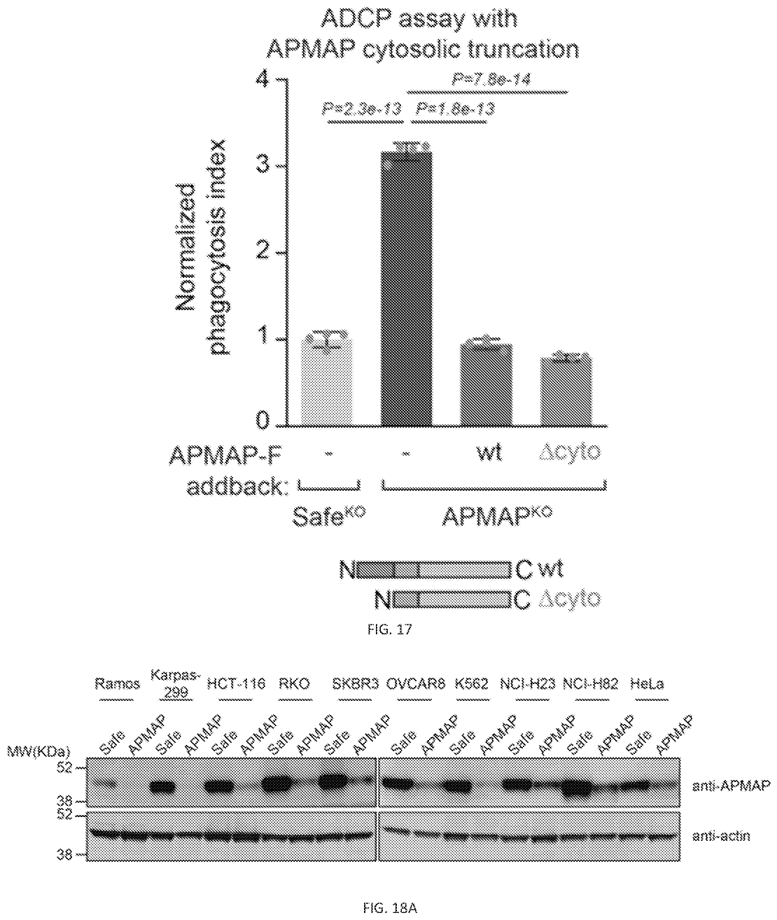

Antibodies that block the interaction of CD47 with the macrophage inhibitory receptor SIRPa are currently under evaluation in multiple clinical trials for use in combination with other immunotherapies and chemotherapeutics. The identification of APMAP as an additional major regulator of tumor susceptibility to ADCP raised the possibility that APMAP loss might synergize with CD47 blockade to further sensitize cancer cells to phagocytosis. Indeed, in an ADCP CRISPR screen in Ramos cells in the presence anti-CD20 antibodies, APMAP was the strongest hit in two screens of a 3,500-gene sublibrary (enriched for cell surface transmembrane proteins) for factors whose deletion synergizes with loss of CD47 function, induced either by blockade with anti-CD47 antibodies (FIG. 2D) or by expression of a CD47-targeting sgRNA (FIGS. 10A-10B). Additionally, in a genome-wide screen for susceptibility to phagocytosis driven by anti-CD47 antibodies in Karpas-299 cells, AIPMAP was among the top hits (FIG. 15B). In quantitative live-cell imaging experiments, APMAP-deficient Ramos and Karpas-299 cells were significantly more sensitive to phagocytosis in the presence of CD47 blocking antibodies than control cells (FIG. 2E). By contrast, in the absence of anti-CD47 antibodies, APMAP-deficient Ramos cells were not more susceptible to phagocytosis than control cells (FIG. 2E), suggesting that at least in this cell line, APMAP loss enhanced ADCP driven by CD47 blockade but did not drive cancer cell uptake in the absence of other phagocytosis-stimulating treatments. As CD47 blockade and rituximab treatment have been previously combined to drive higher rates of ADCP of lymphoma cells, whether APMAP loss synergizes with this treatment combination was tested. Knock out of APMAP enhanced ADCP induced by treatment with anti-CD20 and anti-CD47 antibodies (FIG. 2F). In a series of additional experiments, it was confirmed that APMAP loss specifically sensitized cancer cells to phagocytosis without affecting expression of known pro- or anti-phagocytic signals, binding of tumor-targeting antibodies, or sensitivity to a panel of other cytotoxic agents.

### Example 4

### APMAP Protects Diverse Cancer Cell Lines Against Phagocytosis

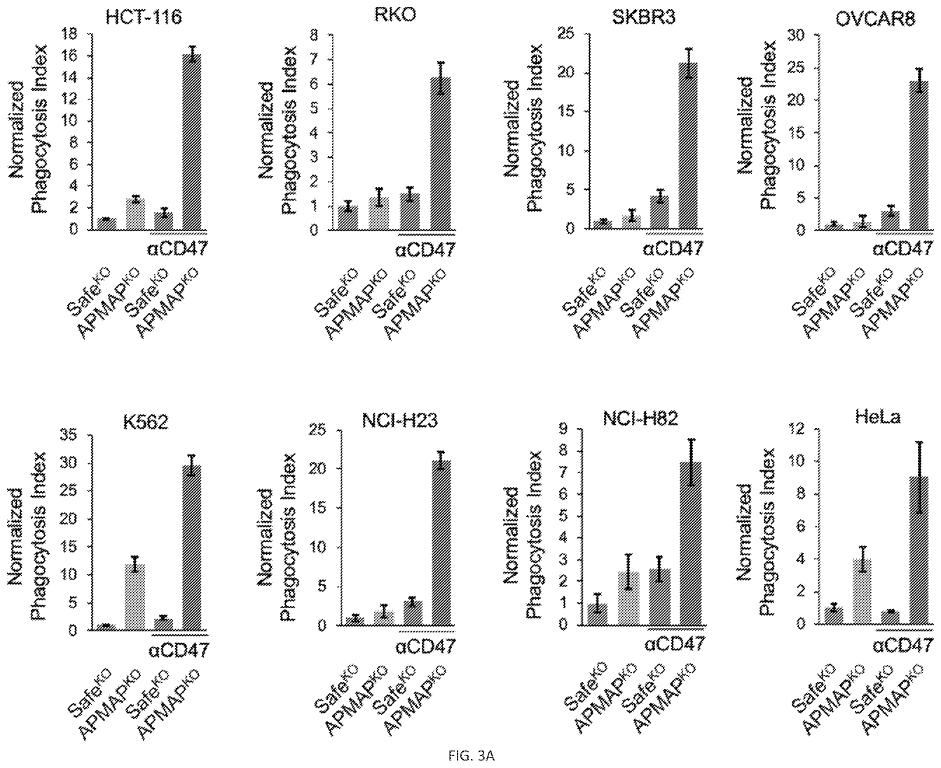

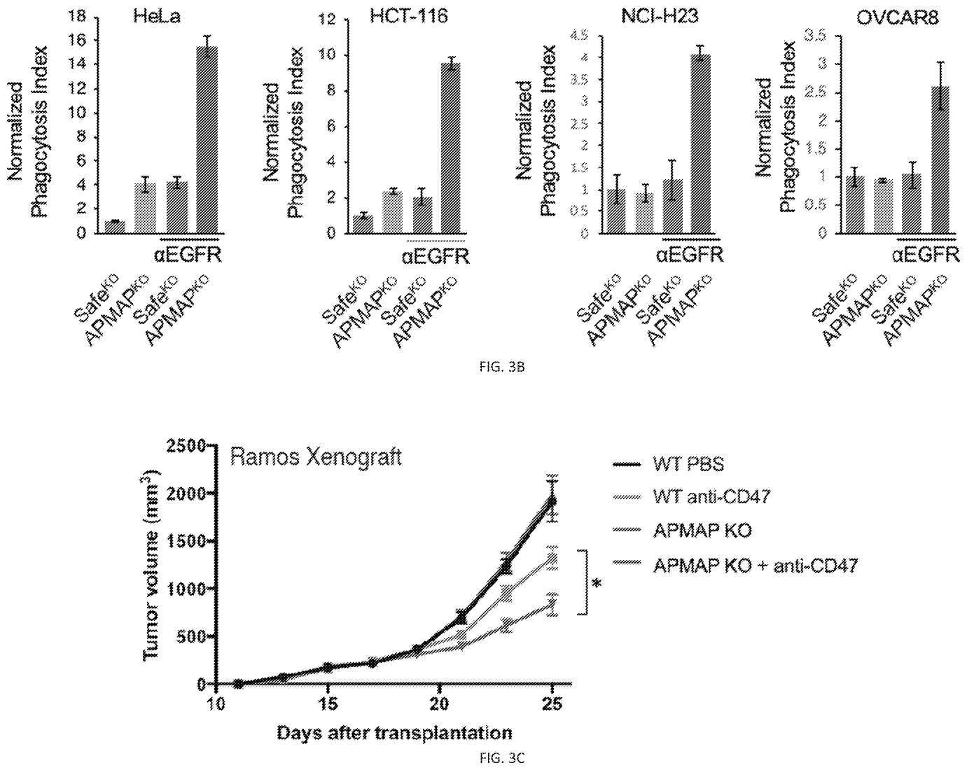

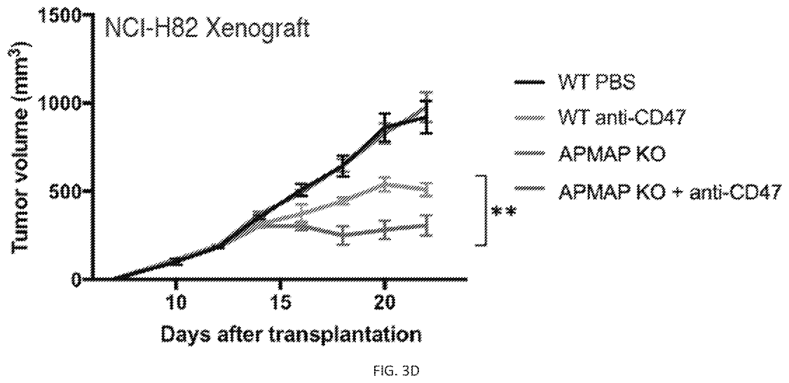

APMAP is widely expressed across many tissue types and is overexpressed in several cancers. To test whether the role for APMAP in protecting cancer cells against phagocytosis uncovered in lymphoma cells is conserved in other tumor types, the sensitivity to phagocytosis of eight additional cancer cell lines derived from diverse tissue types was examined. These cell lines, HeLa, NCI-H23, NCI-H82, RKO, OVCAR8, SKBR3, K562, HCT-116, were derived from cervical, non-small-cell lung cancer, small-cell lung cancer, colon cancer, ovarian cancer, breast cancer, leukemia, and colon cancers, respectively, and express APMAP at moderate levels compared to all cancer cell lines contained in the Cancer Cell Line Encyclopedia. Tumor-antigen targeting antibodies have not been validated for use in driving phagocytosis of all of these cancer cell lines, but each cell line expresses high levels of CD47, enabling induction of phagocytosis with CD47-blocking antibodies. For each of these eight cell lines, expression of APMAP-targeting sgRNAs greatly sensitized cells to phagocytosis in the presence of CD47 blockade (FIG. 3A). Additionally, for the subset of these lines that express EGFR, the target of the therapeutic antibody cetuximab, whether APMAP loss sensitized these lines to ADCP in the presence of this antibody was tested. For HeLa, HCT-116, NCI-H23, and OVCAR8 cells APMAP deficiency increased their susceptibility to phagocytosis driven by cetuximab, even for NCI-H23 and OVCAR8 cells that are not otherwise detectably phagocytosed in the presence of cetuximab (FIG. 3B).

### Example 5

### APMAP Loss Synergizes with CD47 Blockade to Block Tumor Development in Mice

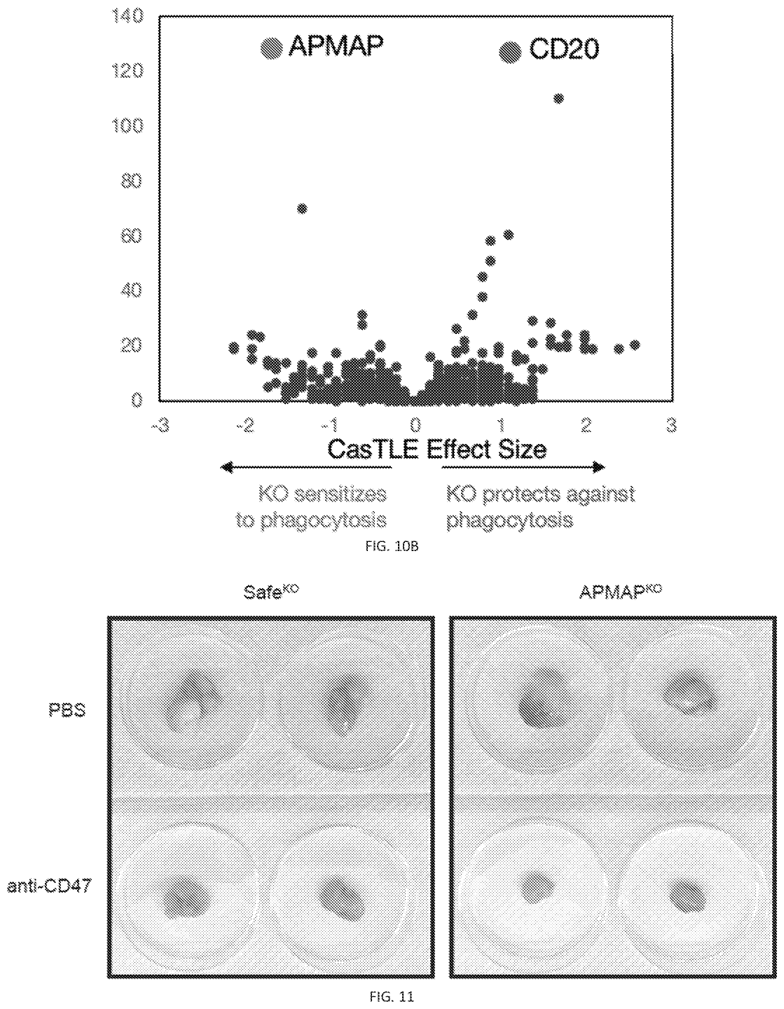

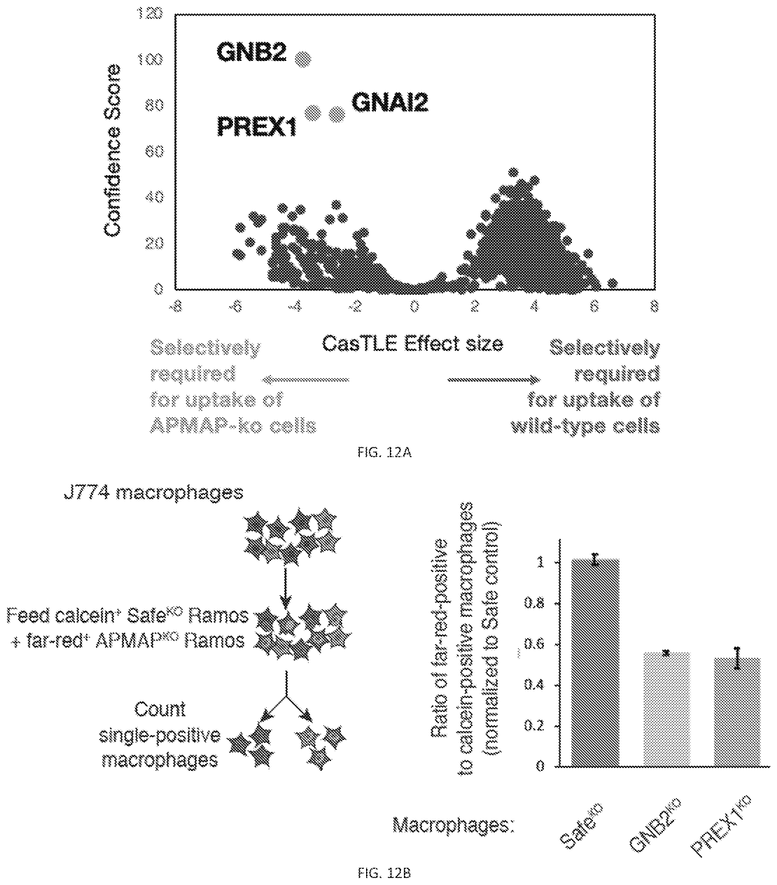

To test whether inhibition of APMAP may represent a clinically viable approach for sensitizing tumors to killing via macrophage-mediated phagocytosis, two preclinical mouse tumor xenograft models, using Ramos lymphoma or NCI-H82 small cell lung cancer tumors, were examined. After allowing tumor xenografts from Safe^{KO} and APMAP^{KO} Ramos and NCI-H82 cells to develop, mice were treated with CD47-blocking antibodies to induce macrophage-mediated killing, or with PBS as a control. In the experiment with NCI-H82 cells, a single pulse of radiation was added two days before initiating CD47 blockade. CD47 blockade inhibited development of both Ramos (FIGS. 3C, 11) and NCI-H82 (FIG. 3D) tumors compared to treatment with PBS. Strikingly, APMAP deficiency enhanced the effect of CD47 blockade on inhibition of the growth of both Ramos and NCI-H82 tumors (FIGS. 3C-3D, 11 and 18G-18H). By contrast, APMAP deficiency had no effect on the development of either tumor type in the absence of CD47 blockade (FIGS. 3C-3D). Thus, APMAP protected cancer cells against macrophage phagocytosis induced by CD47 blockade in a pre-clinical tumor model.

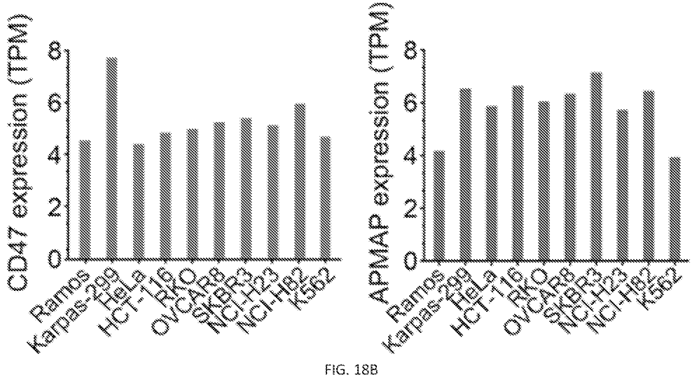

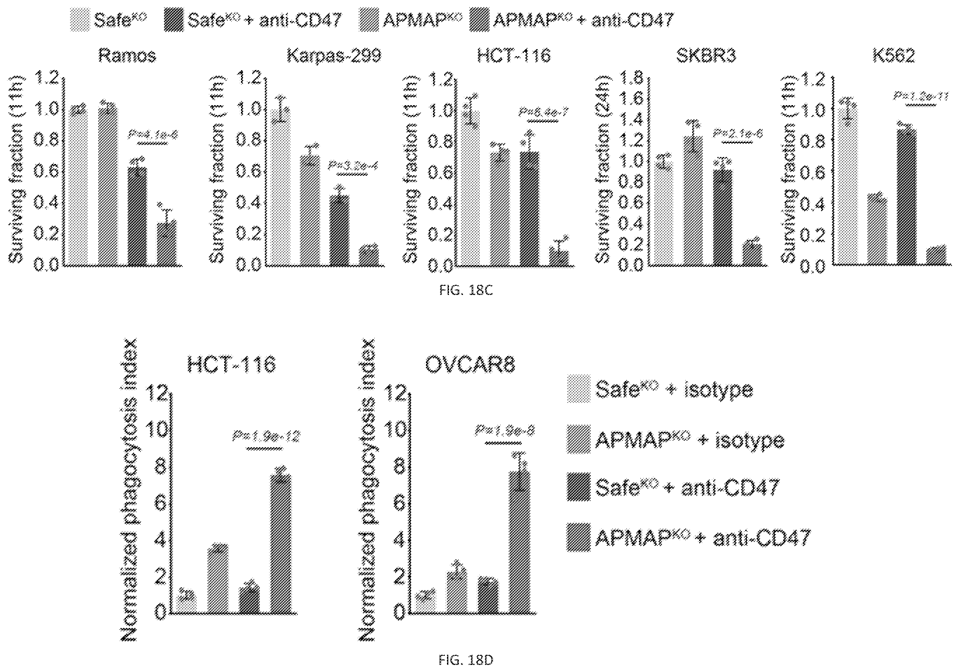

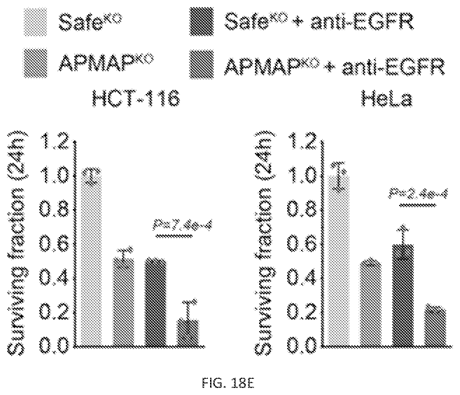

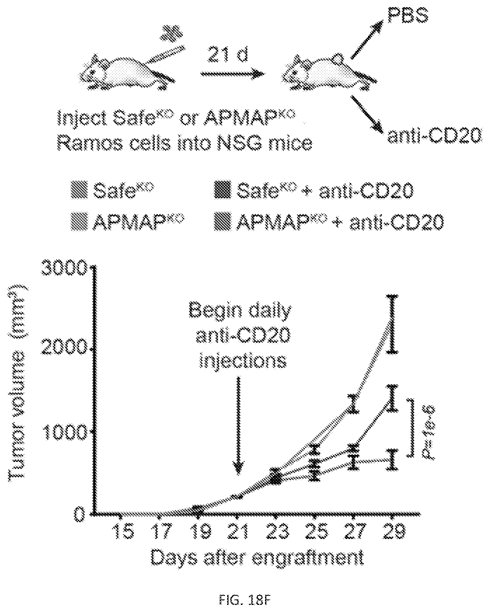

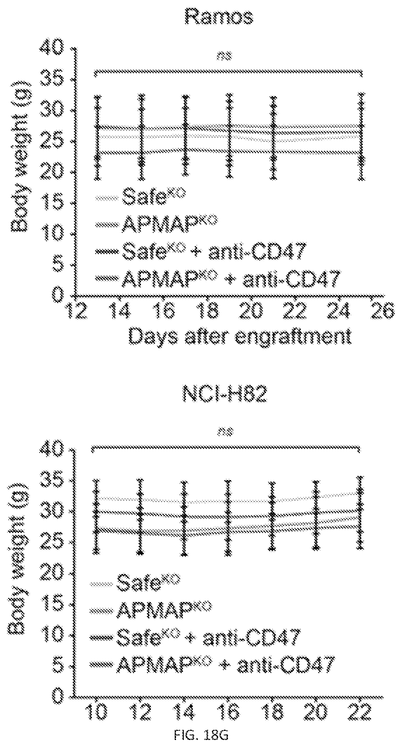

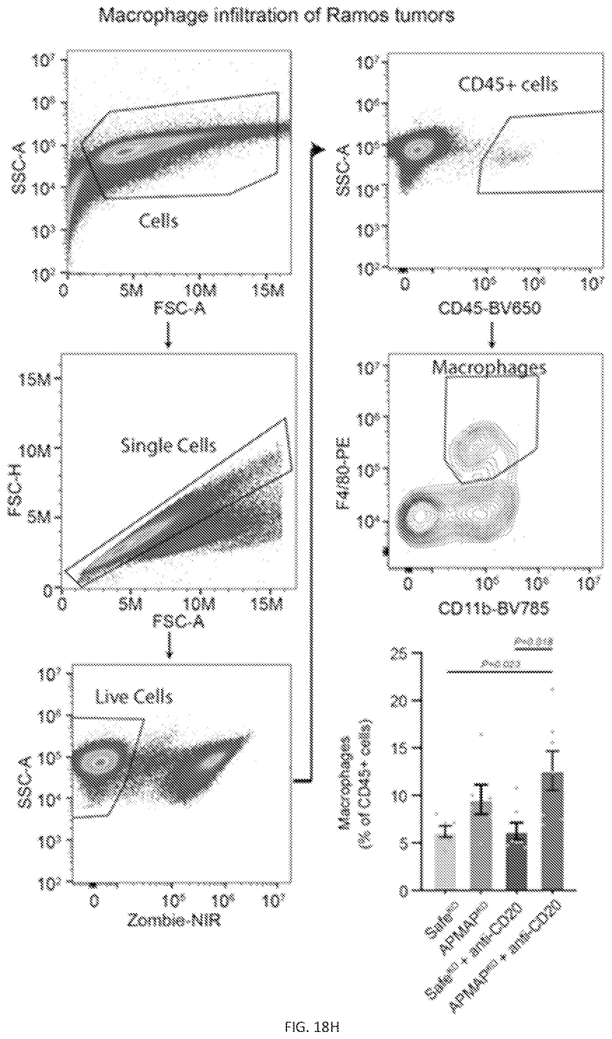

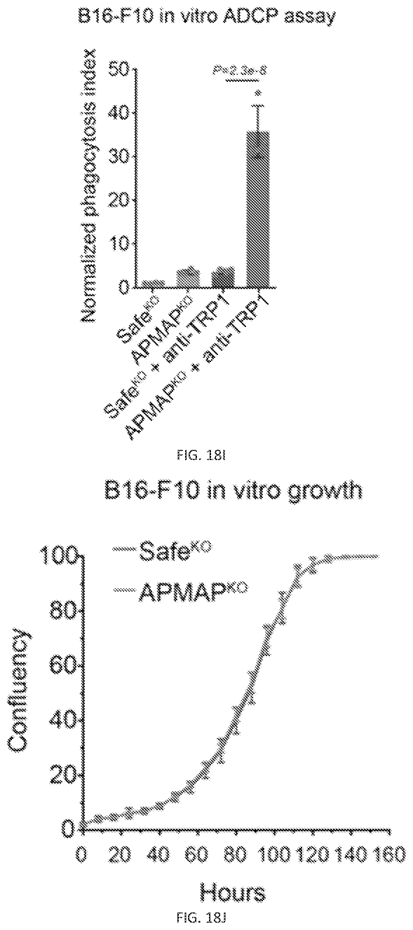

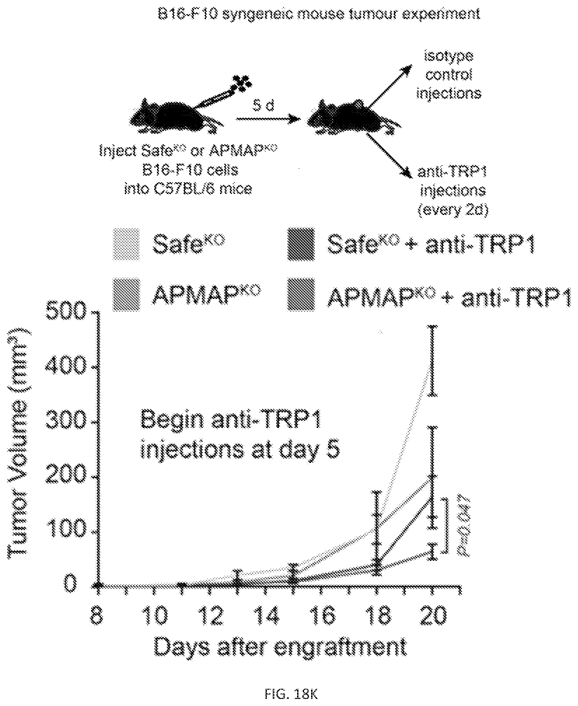

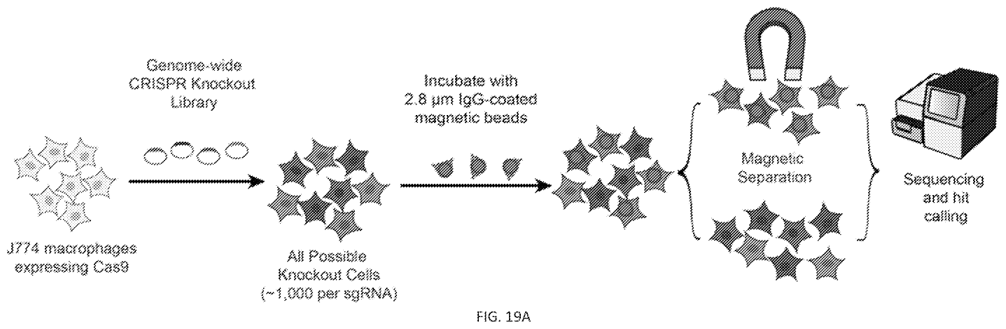

Finally, mouse B16-F10 melanoma cells were injected into syngeneic C57BL/6 mice and treated with anti-TRP1 tumor-targeting antibodies. Tumors lacking APMAP were significantly sensitized to anti-TRP1 treatment (FIGS. 18J-18L). APMAP loss enhanced the effects of multiple tumor-targeting antibodies on inhibition of tumor growth in mice.

### Example 6

### Enhanced Uptake of APMAP^{KO} Cells Requires the Lipid Receptor GPR84

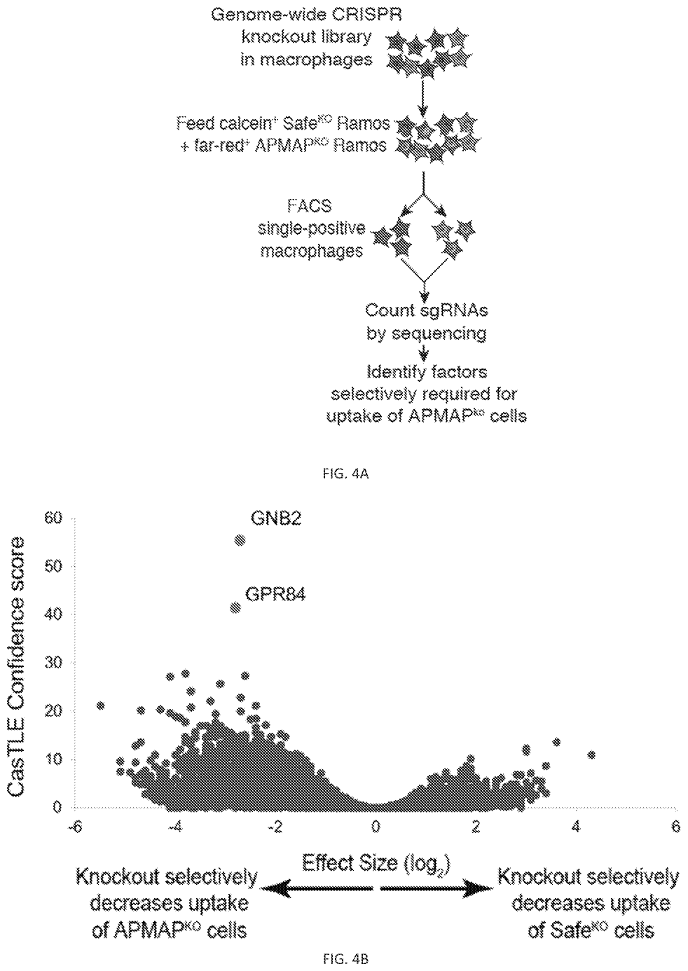

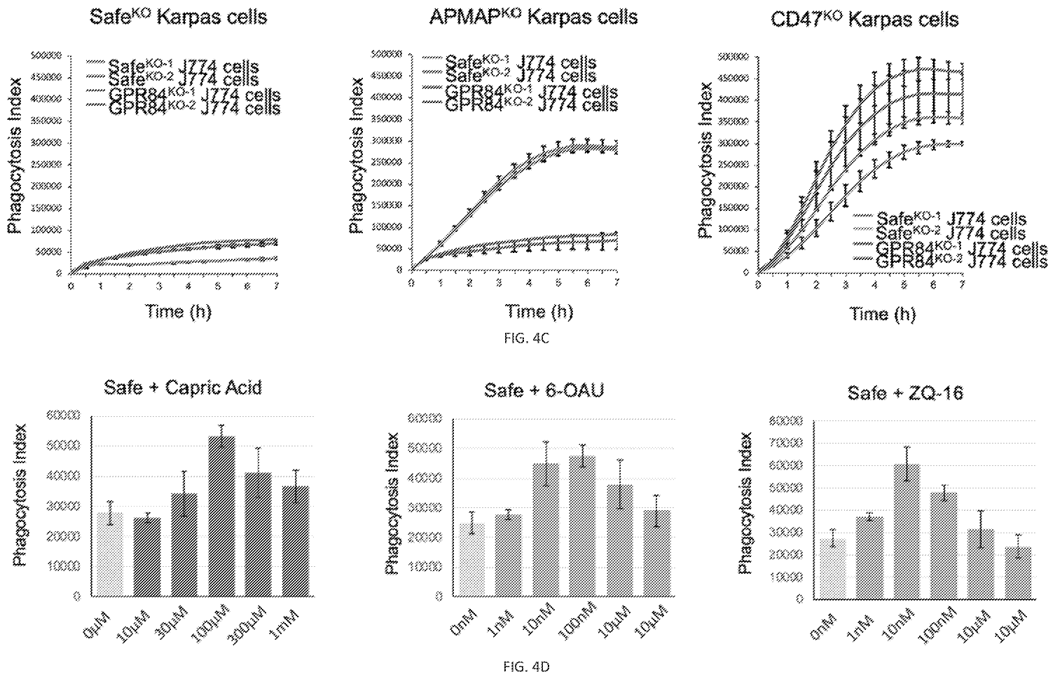

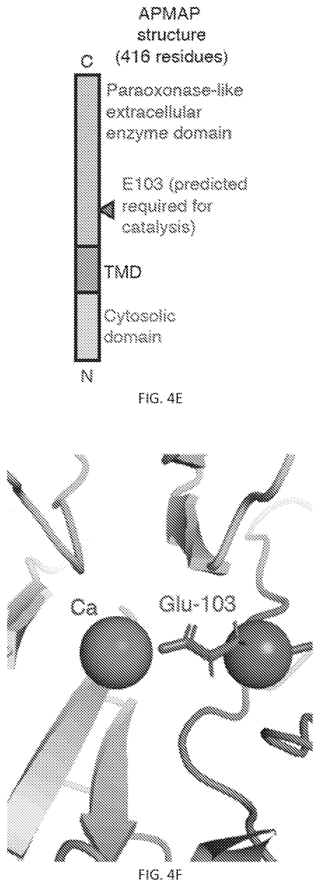

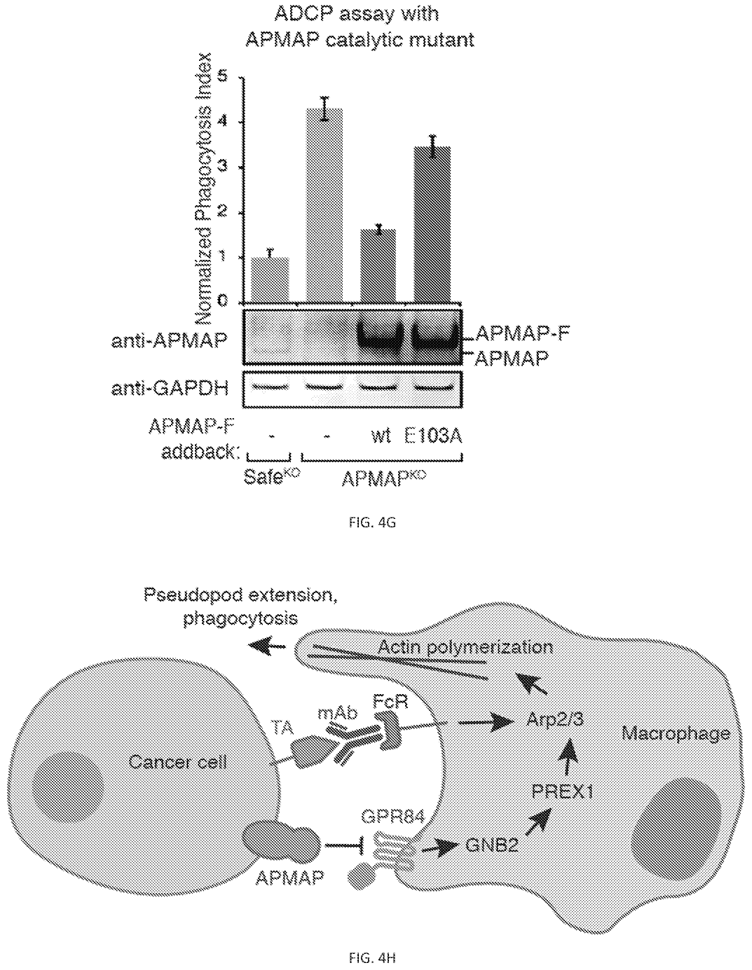

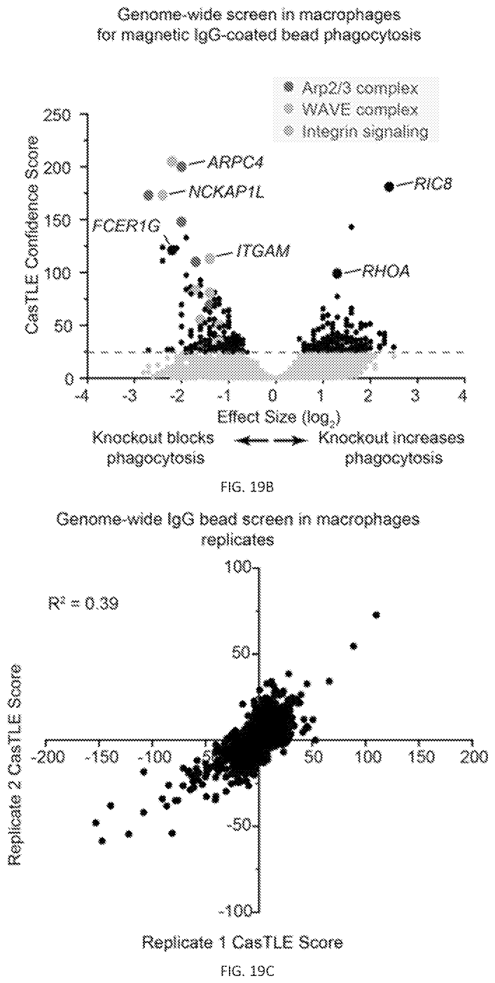

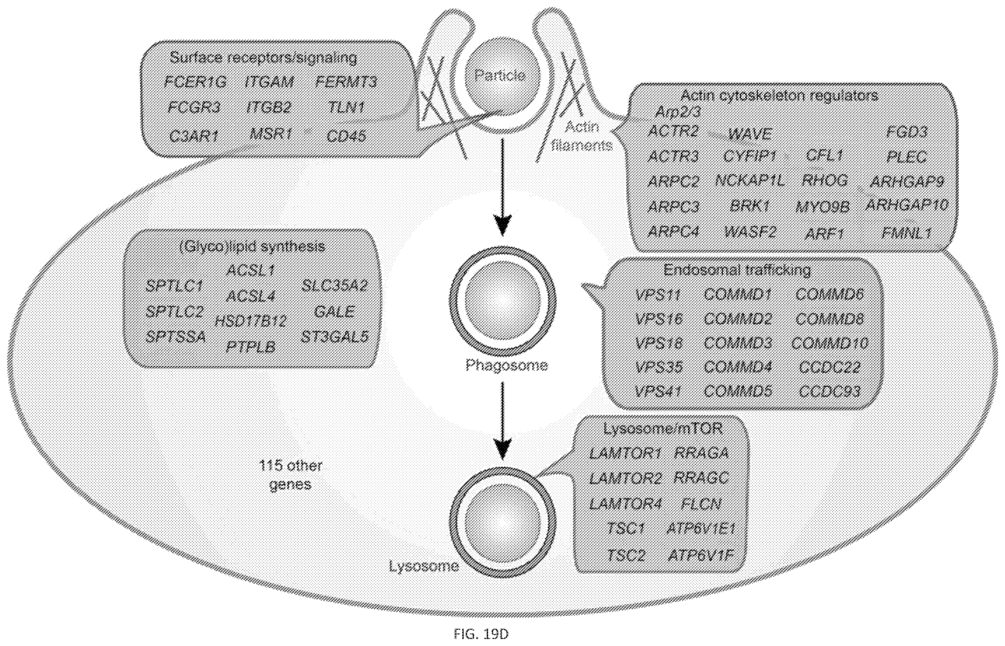

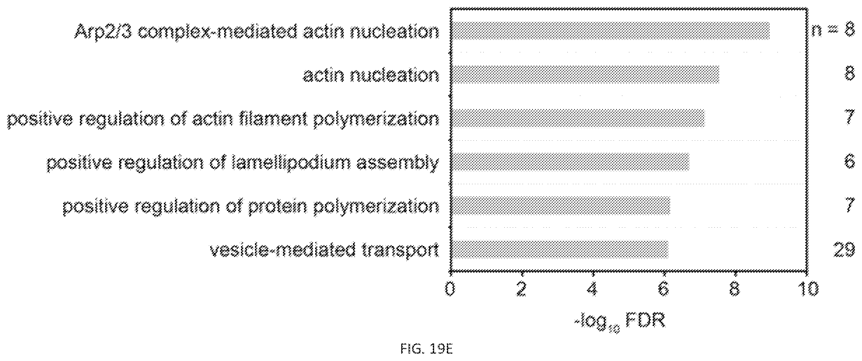

To identify macrophage factors required for enhanced phagocytosis of APMAP^{KO} cells, a FACS-based screen was conducted in which calcein-labeled Safe^{KO} cells and CellTrace Far Red-labeled APMAP^{KO} Ramos cells were fed to a genome-wide knockout macrophage pool (FIGS. 4A and 19). Macrophages were sorted into four populations, and compared sgRNA distributions between the two single-positive populations to identify genes selectively required for uptake of APMAP^{KO} cells, but not Safe^{KO} cells. This analysis revealed two genes as strong hits required specifically for phagocytosis of APMAP^{KO} cells: GPR84 and GNB2. GPR84 is an orphan G-protein coupled receptor that was previously shown to stimulate macrophages phagocytosis when activated, and GNB2 is a heterotrimeric G-beta protein subunit (FIG. 4B).

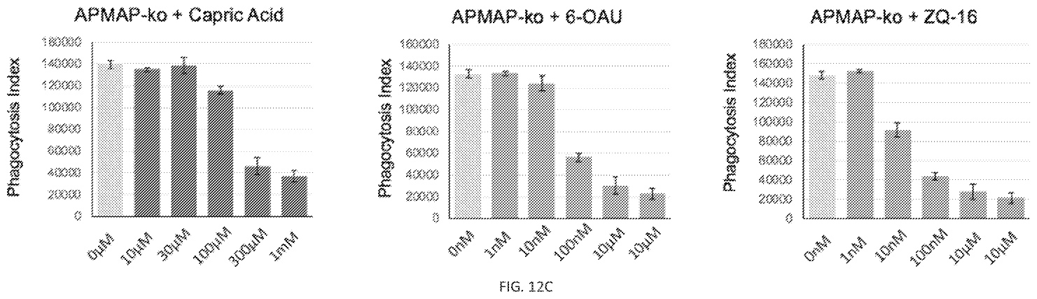

To identify additional genes required for signaling downstream of GPR84, a higher-coverage screen was conducted using a 2,208-gene phagocytosis-regulator enriched sub-library, which additionally revealed a dependence on the G-alpha subunit GNAI2 and the actin regulator PREX1 (FIG. 12A). These macrophage genetic requirements for enhanced ADCP of APMAP^{KO} cells were validated using time-lapse imaging and FACS-based assays, finding that expression of sgRNAs targeting GPR84, GNB2, or PREX1 abolished enhanced uptake of APMAP^{KO} cells, but did not diminish uptake of Safe^{KO} cells (FIGS. 4C and 12B). Additionally, stimulation of GPR84 using either the medium chain fatty acid capric acid, which was previously reported to activate GPR84, or two synthetic agonists, ZQ-16 and 6-OAU, stimulated antibody-dependent phagocytosis of Safe^{KO} cells (FIG. 4D), but, at high concentrations, inhibited uptake of APMAP^{KO} cells (FIG. 12C), which likely reflects GPR84 desensitization following treatment with saturating concentrations of agonists.

### Example 7

### APMAP Catalytic Activity is Required to Protect Cancer Cells Against Phagocytosis

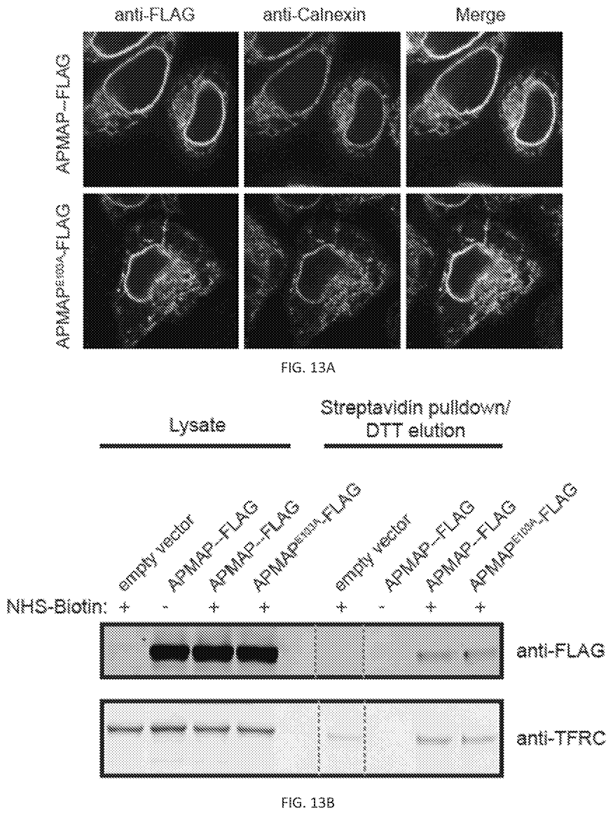

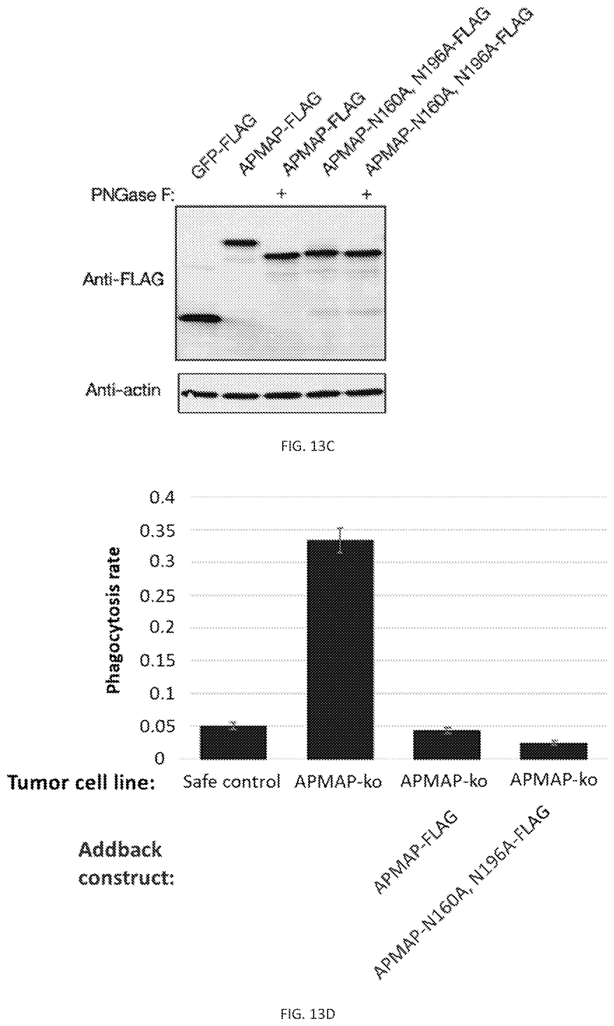

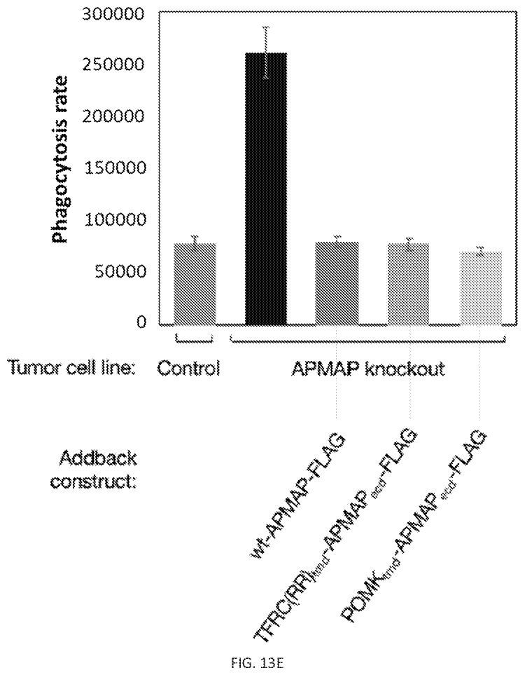

APMAP encodes a 416-amino acid type I membrane protein comprising a short cytosolic domain, a single transmembrane domain, and a predicted extracellular or ER-lumenal domain that exhibits sequence homology with the paraoxonase family of antioxidant enzymes (FIG. 4E). To assess the contributions of each of these domains to APMAP's function in protecting cancer cells against phagocytosis, APMAP^{KO} Ramos cells were transduced with constructs expressing a series of FLAG-tagged APMAP wild-type and mutant constructs. Full-length FLAG-tagged wild-type APMAP, which localized primarily to the endoplasmic reticulum (FIG. 13A), with a small population detected at the cell surface (FIG. 13B), restored the ability of APMAP^{KO} Ramos cells to suppress phagocytosis (FIG. 4G). Truncation of the cytosolic domain, replacement of the transmembrane domain and cytosolic domain of APMAP with that of unrelated ER proteins, or mutation of the two glycosylated residues of APMAP did not affect APMAP function in regulation of ADCP (FIGS. 13C-E). By contrast, expression of a version of APMAP with a mutation in its predicted calcium-binding site that is expected to be required for catalysis based on an APMAP homology model developed using the PON1 structure (FIG. 4F) did not restore APMAP function in the ADCP assay (FIG. 4G), despite exhibiting a similar expression level (FIG. 4G) and localization pattern (FIGS. 13B-C) as wild-type APMAP. Thus, the predicted catalytic activity of APMAP, contained in its ER-luminal/extracellular domain, is required for the ability of APMAP to suppress phagocytosis.

Stimulating the anti-tumor activities of macrophages has recently gained increased attention as a promising therapeutic strategy in cancer immunotherapy, however the suppression of macrophages in the tumor microenvironment by cancer-expressed factors remains a key obstacle. APMAP, as shown herein, was determined as a major regulator of cancer susceptibility to macrophage phagocytosis and, thus, a novel therapeutic target in cancer. Interestingly, unlike previously identified anti-phagocytic factors such as CD47 and CD24, APMAP loss on its own did not affect the susceptibility of most cancer cell lines to phagocytosis, but specifically induced antibody-opsonized cells to be phagocytosed at high rates. It is contemplated that such a synergistic effect is beneficial in a clinical context by decreasing off-target toxicity. Additionally, APMAP is distinct from previously identified cancer inhibitors of macrophage phagocytosis in that it contains an enzymatic domain. An intact catalytic site in this domain facilitated inhibition of ADCP. Importantly, the existence of specific inhibitors of paraoxonase enzymes show that small molecule inhibitors of APMAP function find use to block its role in phagocytosis suppression. Based on homology between APMAP and the paraoxonase family of lipid hydrolases, APMAP may hydrolyze lipids that otherwise activate GPR84, whose activation was shown to be sufficient to stimulate ADCP, thereby preventing cancer cells from triggering phagocytosis by nearby macrophages (FIG. 4H), although an understanding of the mechanism is not necessary to practice the invention. Regardless of these mechanistic possibilities, it is contemplated that combining inhibition of APMAP with both TA-targeting mAbs and/or with blockade of other anti-phagocytic factors, including CD47, PD-L1, and CD24, drives more robust macrophage-mediated control of diverse cancers. Agents and methodologies used to regulate APMAP and/or GPR84 include, but are not limited to, small molecules, antibodies or fragments thereof, aptamers, nucleic acid molecules (e.g., antisense oligonucleotides, siRNAs, etc.), and gene therapy (e.g., knock out or knock in or alteration of expression).

TABLE 1

| | CasTLE |
|---|---|
| Gene | score |
| GFI1 | 696 |
| SMAGP | 503 |
| MUC21 | 475 |
| ST6GALNAC1 | 474 |
| OSR2 | 363 |
| MUC1 | 356 |
| CD47 | 338 |
| GNE | 320 |
| GAL3ST4 | 318 |
| ST3GAL1 | 294 |
| CMAS | 249 |
| LRRC15 | 248 |
| TLE3 | 246 |
| PRDM1 | 229 |
| SPN | 216 |
| MUC12 | 208 |
| PTPRC | 206 |
| HDAC9 | 198 |
| NFIA | 187 |
| NANS | 175 |
| GFI1B | 169 |
| POU2F2 | 162 |
| IRX5 | 162 |
| C1GALT1C1 | 158 |
| QPCTL | 147 |
| SLC35A1 | 145 |
| C5AR1 | 142 |
| CD44 | 133 |
| JMJD1C | 120 |
| CAB39 | 118 |
| UBE2D3 | 115 |
| PODXL | 108 |
| HMHA1 | 104 |
| HIC1 | 102 |
| PDCD10 | 91.9 |
| SLA | 88.9 |
| C1GALT1 | 82.4 |
| POU2AF1 | 81.7 |
| PTEN | 81.3 |
| ZEB2 | 81.2 |
| APMAP | 80.7 |
| SASH3 | 80.7 |
| HES7 | 78.3 |
| BCOR | 78.2 |
| PTPN6 | 77.5 |
| RTN4IP1 | 75.9 |
| RAC2 | 75.6 |
| FOXO4 | 74.9 |
| CAPN6 | 73.7 |
| ST3GAL2 | 71.7 |
| AIFM1 | 69.8 |
| FDX1 | 69.4 |
| CEBPE | 68.8 |
| NDUFA1 | 67 |
| GTPBP6 | 65.2 |
| FCGR1B | 64.5 |
| NDUFS8 | 63.5 |
| TACO1 | 60.1 |
| GPR114 | 59 |
| CMC1 | 58.9 |
| GRHL1 | 58.3 |
| PNMA5 | 58 |
| ATP5SL | 56.7 |
| SLC39A9 | 56.4 |
| SLA2 | 55.6 |
| ZBTB7A | 55.5 |
| CHMP1A | 55 |
| GRSF1 | 54.5 |
| CD79B | 54.3 |
| ZNF683 | 54.3 |
| CIITA | 54.1 |
| ZBTB7B | 53.7 |
| BCL6 | 53.6 |
| C17ORF89 | 53.1 |
| NDUFAF7 | 52.3 |
| PDE12 | 52.2 |
| MAK | 52.1 |
| UQCC1 | 51.2 |
| MAP3K10 | 51.2 |
| NDUFAF5 | 49.4 |
| HIGD2A | 49.1 |
| TMEM119 | 48.5 |
| SIX4 | 48.3 |
| NDUFB9 | 48.1 |
| GYPA | 48.1 |
| ZFX | 47.3 |
| MECR | 47.1 |
| RNF122 | 46.6 |
| MED13 | 45.9 |
| DBR1 | 45.5 |
| MUC22 | 45.3 |
| PWWP2B | 45 |
| WDR1 | 44.8 |
| COX18 | 44.5 |
| DBN1 | 44.5 |
| TTC39C | 43.7 |
| NRG4 | 43.6 |
| MSS51 | 43.4 |
| NDUFS6 | 43.3 |
| FOXO1 | 43.2 |
| TMEM261 | 42.9 |
| VSIG1 | 42.6 |
| SORD | 42.6 |
| DGKB | 42.6 |
| C15ORF59 | 42.4 |
| S100A11 | 42.4 |
| CS | 42.1 |
| ADAD2 | 42 |
| MS4A8 | 42 |
| TRIM13 | 42 |
| POU2F2 | 41.9 |
| ADAM10 | 41.7 |
| NDUFB6 | 41.6 |
| NOMO2 | 41.6 |
| NOMO3 | 41.5 |
| PNMAL1 | 41.4 |
| DOCK10 | 41.3 |
| KCNS2 | 41.3 |
| NOMO1 | 41.3 |
| ZMYND8 | 41.2 |
| SLC30A7 | 41.1 |
| KCNJ6 | 41.1 |
| VPS39 | 41.1 |
| ZNF680 | 41 |
| QSER1 | 40.9 |
| CHAF1A | 40.9 |
| UNC13D | 40.9 |
| IGLL1 | 40.8 |
| TIMM23B | 40.6 |
| MTIF3 | 40.5 |
| PIGR | 40.5 |
| MYH10 | 40.5 |
| NANOS3 | 40.5 |
| MTO1 | 40.2 |
| LPPR1 | 40.1 |
| TIMM23 | 40 |
| UBR4 | 39.9 |
| CD4 | 39.7 |
| KRT23 | 39.6 |
| ARRDC3 | 39.1 |
| RAB44 | 38.9 |
| NDUFB4 | 38.8 |
| JARID2 | 38.7 |
| KRT6A | 38.7 |
| LIPT2 | 38 |
| GK5 | 37.6 |
| MPZL1 | 37.3 |
| HMHA1 | 37 |
| TMPRSS5 | 37 |
| YBEY | 36.7 |
| ZNF521 | 36.4 |
| RDX | 36.4 |
| ARHGAP30 | 36.4 |
| OBP2A | 36.4 |
| ALAD | 36 |
| PCBP4 | 36 |
| NXT1 | 35.8 |
| RPL5 | 35.7 |
| PRKAR1A | 35.3 |
| OTUB1 | 35.2 |
| NDUFV1 | 35.1 |
| GSTM2 | 35 |
| GTPBP3 | 34.8 |
| AMPD2 | 34.7 |
| FXYD5 | 34.7 |
| NUBPL | 34.1 |
| NDUFS2 | 34 |
| NDUFB11 | 33.6 |
| PPT1 | 33.4 |
| ZEB1 | 33.4 |
| ADRBK2 | 33.4 |
| LACTBL1 | 33.3 |
| POLR2H | 33.3 |
| SAMD4B | 32.8 |
| ZBTB7A | 31.9 |
| BTBD19 | 31.8 |
| TIMMDC1 | 31.6 |
| TRIM33 | 31.3 |
| CCNT1 | 31 |
| STARD7 | 30.9 |
| AP000721.4 | 30.9 |
| MAP1B | 30.8 |
| C20ORF166 | 30.8 |
| NFIA | 30.6 |
| SEMA4A | 30.6 |
| UBE2K | 30.4 |
| VPS37A | 30.3 |
| NDUFA9 | 30.2 |
| TMOD1 | 30.1 |
| CEACAM1 | 29.9 |
| COX5B | 29.8 |
| NDUFA8 | 29.8 |
| ESRP1 | 29.7 |
| FBRS | 29.7 |
| CTRC | 29.6 |
| PDK3 | 29.5 |
| PTPRC | 29.2 |
| ACTB | 29.1 |
| NDUFAF3 | 29.1 |
| FGD3 | 29 |
| HMBS | 28.9 |
| NDUFC1 | 28.9 |
| GMIP | 28.6 |
| BHLHA15 | 28.4 |
| TMEM38B | 28.2 |
| LYN | 28.1 |
| NDUFS7 | 27.8 |
| B3GNT7 | 27.8 |
| FEZ2 | 27.8 |
| MRPS2 | 27.6 |
| PRKCD | 27.5 |
| MYCBP2 | 27.5 |
| FLI1 | 27.5 |
| TBX22 | 27.5 |
| VPS37C | 27.5 |
| STUB1 | 27.3 |
| NDUFS1 | 27 |
| SMS | 26.9 |
| MRPL24 | 26.7 |
| AHR | 26.7 |
| LIPT1 | 26.5 |
| NLRC3 | 26.5 |
| SORCS1 | 26.3 |
| SCYL1 | 26.2 |
| CLCC1 | 26.2 |
| RPP14 | 26.1 |
| XRN1 | 26 |
| ZP2 | 25.9 |
| OXER1 | 25.9 |
| LENG9 | 25.8 |
| C1QBP | 25.7 |
| MRPL37 | 25.7 |
| UBE2E2 | 25.7 |
| TBC1D22A | 25.6 |
| NOP58 | 25.5 |
| CR2 | 25.5 |
| KCMF1 | 25.4 |
| COQ9 | 25.4 |
| IRF2 | 25.4 |
| MXRA5 | 25.3 |
| TOMM70A | 25.1 |
| NDUFAF6 | 25.1 |
| PLEKHO2 | 25.1 |

TABLE 2

| Gene | Gene | Gene |
|---|---|---|
| HSD17B12 | MYC | SF3B14 |
| DOCK2 | RUVBL1 | DUX4L5 |
| CAPRIN1 | VTA1 | RRP9 |
| STAG2 | VPS26A | MRPS2 |
| GSK3A | ACTR1A | LIN54 |
| CFLAR | PCSK7 | OXSM |
| RBM12 | SEC23B | NDUFA6 |
| BCLAF1 | ZNF281 | NDUFB7 |
| ELAVL1 | ARPC3 | CIT |
| SSR4 | PAG1 | SUV420H1 |
| FBXW7 | ATP5C1 | NDUFB8 |
| LIAS | PHACTR4 | PSMD3 |
| ARL1 | C19ORF43 | RP11-234B24.6 |
| SRSF10 | SYMPK | NPDC1 |
| PPIH | SZRD1 | PDSS2 |
| CCDC6 | MEF2BNB-MEF2B | NAPA |
| COPG1 | RPP21 | NIPBL |
| FAM58A | SDHC | EIF3B |
| EEF1A1 | INTS5 | PEPD |
| ST6GAL1 | ARID2 | COX20 |
| PPP1R2 | COA3 | MYB |
| USP7 | PARS2 | C1ORF233 |
| CLASRP | PTPMT1 | RRAGC |
| PTAR1 | COPB2 | SHQ1 |
| CDK6 | DDXSS | UBE3D |
| GMDS | TRA2A | NDUFA2 |
| HECTD1 | VPS72 | IER5L |
| SPPL3 | | |
| NDUFS5 | | |
| IKZF3 | | |
| UBE2J2 | | |
| PPOX | | |
| IDH3B | | |
| CYTH1 | | |
| NDUFB10 | | |
| TMEM9B | | |
| WDR26 | | |
| YPEL5 | | |
| ZBTB16 | | |
| PTOV1 | | |

TABLE 3

| | Inhibitor, Agonist, or | |
|---|---|---|
| Gene | Antagonists | Supplier or Reference |
| PTEN | VO-Ohpic trihydrate | SelleckChem |
| GSK3A | CHIR-98014 | SelleckChem |
| ADAM10 | GI 254023X | Tocris |
| HSD17B12 | INH-12 | Mohamed et al., Sci Rep 10, 4040 |
| | | (2020), incorporated herein by |
| | | reference in its entirety |
| HDAC9 | TMP195 | SelleckChem |
| CDK6 | BSJ-03-123 | SelleckChem |
| C5AR1 | PMX53 | Tocris |

It is understood that the foregoing detailed description and accompanying examples are merely illustrative and are not to be taken as limitations upon the scope of the disclosure, which is defined solely by the appended claims and their equivalents.

All publications and patents mentioned in the above specification are herein incorporated by reference as if expressly set forth herein. Various changes and modifications to the disclosed embodiments will be apparent to those skilled in the art and may be made without departing from the spirit and scope thereof.

1. A method of treating a disease or disorder or sensitizing a cell to phagocytosis comprising contacting a cell with:
(a) an inhibitor of an anti-phagocytic gene selected from the group consisting of: GFI1; SMAGP; MUC21; ST6GALNAC1; OSR2; MUC1; CD47; GNE; GAL3ST4; ST3GAL1; CMAS; LRRC15; TLE3; PRDM1; SPN; MUC12; PTPRC; HDAC9; NFIA; NANS; GFI1B; POU2F2; IRX5; C1GALT1C1; QPCTL; SLC35A1; C5AR1; CD44; JMJD1C; CAB39; UBE2D3; PODXL; HMHA1; HIC1; PDCD10; SLA; C1GALT1; POU2AF1; PTEN; ZEB2; APMAP; SASH3; HES7; BCOR; PTPN6; RTN4IP1; RAC2; FOXO4; CAPN6; ST3GAL2; AIFM1; FDX1; CEBPE; NDUFA1; GTPBP6; FCGR1B; NDUFS8; TACO1; GPR114; CMC1; GRHL1; PNMA5; ATP5SL; SLC39A9; SLA2; ZBTB7A; CHMP1A; GRSF1; CD79B; ZNF683; CIITA; ZBTB7B; BCL6; C17ORF89; NDUFAF7; PDE12; MAK; UQCC1; MAP3K10; NDUFAF5; HIGD2A; TMEM119; SIX4; NDUFB9; GYPA; ZFX; MECR; RNF122; MED13; DBR1; MUC22; PWWP2B; WDR1; COX18; DBN1; TTC39C; NRG4; MSS51; NDUFS6; FOXO1; TMEM261; VSIG1; SORD; DGKB; C15ORF59; S100A11; CS; ADAD2; MS4A8; TRIM13; POU2F2; ADAM10; NDUFB6; NOMO2; NOMO3; PNMAL1; DOCK10; KCNS2; NOMO1; ZMYND8; SLC30A7; KCNJ6; VPS39; ZNF680; QSER1; CHAF1A; UNC13D; IGLL1; TIMM23B; MTIF3; PIGR; MYH10; NANOS3; MTO1; LPPR1; TIMM23; UBR4; CD4; KRT23; ARRDC3; RAB44; NDUFB4; JARID2; KRT6A; LIPT2; GK5; MPZL1; HMHA1; TMPRSS5; YBEY; ZNF521; RDX; ARHGAP30; OBP2A; ALAD; PCBP4; NXT1; RPL5; PRKAR1A; OTUB1; NDUFV1; GSTM2; GTPBP3; AMPD2; FXYD5; NUBPL; NDUFS2; NDUFB11; PPT1; ZEB1; ADRBK2; LACTBL1; POLR2H; SAMD4B; ZBTB7A; BTBD19; TIMMDC1; TRIM33; CCNT1; STARD7; AP000721.4; MAP1B; C20ORF166; NFIA; SEMA4A; UBE2K; VPS37A; NDUFA9; TMOD1; CEACAM1; COX5B; NDUFA8; ESRP1; FBRS; CTRC; PDK3; PTPRC; ACTB; NDUFAF3; FGD3; HMBS; NDUFC1; GMIP; BHLHA15; TMEM38B; LYN; NDUFS7; B3GNT7; FEZ2; MRPS2; PRKCD; MYCBP2; FLI1; TBX22; VPS37C; STUB1; NDUFS1; SMS; MRPL24; AHR; LIPT1; NLRC3; SORCS1; SCYL1; CLCC1; RPP14; XRN1; ZP2; OXER1; LENG9; C1QBP; MRPL37; UBE2E2; TBC1D22A; NOP58; CR2; KCMF1; COQ9; IRF2; MXRA5; TOMM70A; NDUFAF6; PLEKHO2; HSD17B12; and combinations thereof;
(b) an inhibitor of an anti-phagocytic gene selected from the group consisting of: DOCK2; CAPRIN1; STAG2; GSK3A; CFLAR; RBM12; BCLAF1; ELAVL1; SSR4; FBXW7; LIAS; ARL1; SRSF10; PPIH; CCDC6; COPG1; FAM58A; EEF1A1; ST6GALL; PPP1R2; USP7; CLASRP; PTAR1; CDK6; GMDS; HECTD1; MYC; RUVBL1; VTA1; VPS26A; ACTR1A; PCSK7; SEC23B; ZNF281; ARPC3; PAG1; ATP5C1; PHACTR4; C19ORF43; SYMPK; SZRD1; MEF2BNB-MEF2B; RPP21; SDHC; INTS5; ARID2; COA3; PARS2; PTPMT1: COPB2; DDX55; TRA2A; VPS72; SF3B14; DUX4L5; RRP9; MRPS2; LIN54; OXSM; NDUFA6; NDUFB7; CIT; SUV420H1; NDUFB8; PSMD3; RP11-234B24.6; NPDC1; PDSS2; NAPA; NIPBL; EIF3B; PEPD; COX20; MYB; C1ORF233; RRAGC; SHQ1; UBE3D; NDUFA2; IER5L; SPPL3; NDUFS5; IKZF3; UBE2J2; PPOX; IDH3B; CYTH1; NDUFB10; TMEM9B; WDR26; YPEL5; ZBTB16; PTOV1; and combinations thereof; or
(c) an inhibitor of Adipocyte Plasma Membrane Associated Protein (APMAP), an antagonist of SMAGP, an agonist of fatty-acid G-protein coupled receptor GPR84, or combinations thereof.

2-4. (canceled)

5. The method of claim 1, wherein the agonist of fatty-acid G-protein coupled receptor GPR84;
(a) comprises a lipid or a synthetic agonist;
(b) comprises a lipid comprising medium chain fatty acid capric acid; or
(c) is selected from the group consisting of ZO-16, (octylamino) pyrimidine-2,4(1H,3H)-dione (6-n-octylaminouracil, 6-OAU), and combinations thereof.

6-7. (canceled)

8. The method of claim 1, further comprising contacting the cell with at least one or both of:
(a) a tumor antigen (TA)-targeting antibody or other antibody; and
(b) a CD47 blocking antibody or blocking agent.

9. The method of claim 8, wherein the TA-targeting antibody or other antibody comprises rituximab, ibritumomab, obinutuzumab, ofatumumab, tositumomab, gemtuzumab, alemtuzumab, brentuximab, cetuximab, necitumumab, panitumumab, bevacizumab, ipilimumab, pertuzumab, trastuzumab, blinatumomab, or combinations thereof.

10. The method of claim 8, wherein the CD47 blocking antibody or blocking agent comprises an anti-CD47 antibody, an anti-SIRPalpha antibody, a soluble SIRPalpha fragment, or any combination thereof.

11. The method of claim 1, further comprising contacting the cell with an inhibitor of an anti-phagocytic factor.

12. The method of claim 11, wherein the anti-phagocytic factor is selected from the group consisting of PD-L1, CD24, or combinations thereof.

13. The method of claim 1, wherein the disease or disorder comprises an autoimmune disorder, cancer, or atherosclerosis.

14. The method of claim 13, wherein the cell is a cancer cell.

15. The method of claim 13 or 14, wherein the cancer or cancer cell:
(a) is resistant to antibody-dependent cellular phagocytosis (ADCP);
(b) overexpresses CD47;
(c) is a solid tumor; or
(d) comprises lymphoma, cervical cancer, lung cancer, colorectal cancer, ovarian cancer, breast cancer and/or leukemia.

16-18. (canceled)

19. The method of claim 13, wherein the autoimmune disorder comprises rheumatoid arthritis or multiple sclerosis.

20. The method of claim 1, wherein contacting the cell comprises administration to a subject in need thereof.

21. A method for identifying regulators of antibody-dependent cellular phagocytosis (ADCP) in cells comprising:
(i) incubating cells with LPS-treated macrophages in the presence of anti-CD20, anti-EGFR, anti-CD30, and/or anti-CD47 antibodies, wherein the cells comprise a CRISPR knockout system or a CRISPR activation (CRISPRa) system and each cell comprises at least one guide RNA targeting an endogenous gene;
(ii) separating unphagocytosed cells from macrophages;
(iii extracting nucleic acids from the unphagocytosed cells; and
(iv) identifying the guide RNA and guide RNA endogenous gene targets in the unphagocytosed cells.

22. The method of claim 21, wherein:
(a) the cells are cancer cells, lymphoma cells, or Ramos or Karpas-299 lymphoma cells;
(b) the macrophages are J774 macrophage cells;
(c) the macrophages are treated with 10 ng/mL LPS 24 hours prior to incubation with the cells;
(d) the cells comprise a CRISPR knockout system and the incubation is in the presence of anti-CD20 antibodies; or
(e) the cells comprise a CRISPRa system and the incubation is in the presence of anti-CD20 and anti-CD47 antibodies.

23-28. (canceled)

29. The method of claim 21, wherein steps (i) and (ii) are repeated at least once prior to step (iii).

30. The method of claim 21, wherein the identifying comprises sequencing the guide RNA.

31. A method for identifying regulators of phagocytosis in macrophages comprising:
(i) incubating cells comprising a detectable label with LPS-treated macrophages, wherein the macrophages comprise a CRISPR knockout system or a CRISPR activation (CRISPRa) system and each macrophage comprises at least one guide RNA targeting an endogenous gene;
(ii) removing unphagocytosed cells from macrophages; and
(iii) separating the macrophages based on presence or absence of the detectable label.

32. The method of claim 31, wherein:
(a) the cells are cancer cells, lymphoma cells, or Ramos or Karpas-299 lymphoma cells;
(b) the cells lack a regulator of phagocytosis;
(c) the detectable label comprises a fluorescent label;
(d) the macrophages are J774 macrophage cells; and/or
(e) the macrophages are treated with 10 ng/mL LPS 24 hours prior to incubation with the cells.

33-38. (canceled)

39. The method of claim 8, wherein the TA-targeting antibody or other antibody targets CD20, CD33, CD52, CD30, EGFR, VEGF, CTLA-4, EpCAM, PD-1, PD-L1, or combinations thereof.
